(19) United States
(12) Patent Application Publication
(10) Pub. No.: US 2026/0014315 A1
(43) Pub. Date: Jan. 15, 2026

(54) SYSTEM AND METHOD FOR A SELF-PRIMING SYSTEM FOR THE INFUSION OF A LIQUID INTO SUBCUTANEOUS TISSUES

(71) Applicant: KORU Medical Systems, Inc., Mahwah, NJ (US)

(72) Inventors: SUMANTH CHAKRAVARTHY, Flemington, NJ (US); ADAM EDELHAUSER, Kinnelon, NJ (US); BRIAN CHRISTOPHER CASE, Lake Villa, IL (US)

(73) Assignee: KORU Medical Systems, Inc., Mahwah, NJ (US)

(21) Appl. No.: 19/332,576

(22) Filed: Sep. 18, 2025

Related U.S. Application Data
(63) Continuation of application No. 18/760,544, filed on Jul. 1, 2024.

(51) Int. Cl. A61M 5/158; A61M 5/142; A61M 39/22

(52) U.S. Cl. CPC A61M 5/158; A61M 5/142; A61M 39/22; A61M 2205/02

(57) ABSTRACT

Provided is a vented needle hub assembly for infusion of a liquid into subcutaneous tissue. More specifically, the vented needle hub assembly is provided by a needle hub having an inlet side and an outlet side and a fluid passageway therebetween; an infusion needle in fluid contact with the fluid passageway and extending from the outlet side; a flexible tubing having a first end joined to the inlet side in fluid contact with the fluid passageway, and a second end; at least one vent disposed in a sidewall of the needle hub, the vent in fluid contact with the fluid passageway; and a porous plug disposed within each at least one vent adjacent to the fluid passageway, the porous plug structured and arranged to distinguish gas from liquid, gas permitted to pass from the fluid passageway through the porous plug and out the at least one vent; and a reservoir of liquid provided as an autoinjector, the second end of the flexible tubing interconnecting the reservoir of liquid to the inlet side for fluid communication from the reservoir to the needle hub. An associated method of use is provided as well.

### CROSS REFERENCE TO RELATED APPLICATIONS

The present invention is a continuation of U.S. patent application Ser. No. 18/760,544 filed Jul. 1 12, 2016, entitled SYSTEM AND METHOD FOR VENTED NEEDLE HUB FOR INFUSION now U.S. Pat. No. ______, and incorporated herein by reference. This continuing application claims the benefit of U.S. patent application Ser. No. 15/291,895.

### FIELD OF THE INVENTION

The present invention relates generally to medical devices, and more specifically to a vented needle, or needle hub, as used for infusion, the vent permitting the escape of air from the infusion line prior to the delivery of liquid into the patient.

### BACKGROUND

Liquid pharmaceuticals are commonly delivered to patients through injection or infusion. With either type of delivery, it is generally preferable to eliminate air from the delivery system. With a syringe and basic injection, the operator is generally skilled and the amount of air to be purged may be very little-some in the syringe as well as the needle, and a simple depression of the plunger easily disgorges the air from both, with the user releasing pressure upon the plunger as soon as liquid is observed.

With infusion systems, the process can be more trying. Air may exist in both the reservoir from which the liquid is provided-often a syringe, but also throughout the lengths of infusion tubing and their associated needles. With subcutaneous infusion systems, it is not uncommon for multiple needles to be disposed into the patient receiving therapy, and as each tubing and needle element is distinct, each has its own initial volume of air or sterile gas.

Typically, an operator will turn on the infusion pump and drive liquid throughout the system until liquid is observed at each needle. But this can be problematic in several ways. With home infusion therapy care, the operator may not be a true medical practitioner, and he or she may not be quick to realize that the system has been purged, thus leading to excess runoff at the needles which may make insertion messy, and potentially difficult for the needles to be temporarily affixed to the skin as it is slick with liquid. In addition, some medications can cause irritation, if not pain, if disposed upon a person's skin. The operator may also be slow to shut off the pump leading to excessive loss of treatment liquid, which of course could lead to a shortage of the intended dose.

With intravenous infusion, the issue of air or gas introduced through a needle can have immediate and life-threatening consequences. Although subcutaneous infusion is as the name infers, not performed by injecting the liquid into a vein or other primary blood circulation structure, the introduction of air or gas into the subcutaneous tissues can be discomforting and undesirable.

Thus, for at least the issues of mitigating loss of treatment liquid, ensuring proper dosage, adhesion of the needles to the surface of the injection site, ease and comfort of the patient, and of course allowing the administrating party to place the needles and instigate the treatment session with ease and their own sense of comfort, the issue of purging air or sterile gas from an infusion system can be stressful to both the patient and the care giver.

Hence there is a need for a vented needle hub to overcome one or more of the above identified challenges.

### SUMMARY OF THE INVENTION

Our invention solves the problems of the prior art by providing novel systems and methods for a vented needle hub for infusion.

In particular, and by way of example only, according to one embodiment of the present invention, provided is a needle hub having an inlet side and an outlet side and a fluid passageway therebetween; an infusion needle in fluid contact with the fluid passageway and extending from the outlet side; a flexible tubing having a first end joined to the inlet side in fluid contact with the fluid passageway, and a second end; at least one vent disposed in a sidewall of the needle hub, the vent in fluid contact with the fluid passageway; and a porous plug disposed within each at least one vent adjacent to the fluid passageway, the porous plug structured and arranged to allow gas to pass while blocking liquid, gas permitted to pass from the fluid passageway through the porous plug and out the at least one vent; and a reservoir of liquid provided as an autoinjector, the second end of the flexible tubing interconnecting the reservoir of liquid to the inlet side for fluid communication from the reservoir to the needle hub.

In yet another embodiment, provided is a needle hub having a tubing inlet at a first location and a needle extending from a second location, and at least one sidewall; a fluid passageway between the tubing inlet and the needle; at least one vent in the needle hub between the first location and the second location, the at least one vent in fluid communication with the fluid passageway; and a porous plug disposed within each at least one vent adjacent to the fluid passageway; and a reservoir of liquid provided as an autoinjector; a flexible tubing having a first end coupled to the tubing inlet and a second end for interconnecting the reservoir of liquid to the tubing inlet for fluid communication from the reservoir to the needle hub; wherein the porous plug has a first air flow resistance lower than a second air flow resistance of subcutaneous tissue, and a first liquid flow resistance higher than second liquid flow resistance of subcutaneous tissue.

For yet another embodiment, provided is a needle hub having a first end and a second end and a fluid passageway therebetween, the first end joined with a first end of a flexible tubing line and the second end joined with a needle; at least one vent in the needle hub between the first end and the second end, the at least one vent in fluid communication with the fluid passageway; a porous plug disposed within each at least one vent adjacent to the fluid passageway, the porous plug structured and arranged to permit gas to pass from the fluid passageway through the porous plug and out the at least one vent; a reservoir of liquid provided as an autoinjector, the flexible tubing having a second end for interconnecting the reservoir of liquid to the needle hub for fluid communication from the reservoir to the needle hub.

Further still, in yet another embodiment provided is a method of using a self-priming system for the infusion of a liquid into subcutaneous tissue for infusion, including: a needle hub having an inlet side and an outlet side and a fluid passageway therebetween; an infusion needle in fluid contact with the fluid passageway and extending from the outlet side; a flexible tubing having a first end joined to the inlet side in fluid contact with the fluid passageway, and a second end; at least one vent disposed in a sidewall of the needle hub, the vent in fluid contact with the fluid passageway; and a porous plug disposed within each at least one vent adjacent to the fluid passageway, the porous plug structured and arranged to distinguish gas from liquid, gas permitted to pass from the fluid passageway through the porous plug and out the at least one vent; and a reservoir of liquid provided as an autoinjector, the second end of the flexible tubing interconnecting the reservoir of liquid to the inlet side for fluid communication from the reservoir to the needle hub; disposing the needle into the subcutaneous tissue of a patient; and activating the autoinjector to dispense liquid from the reservoir into the flexible tubing, the liquid propagating the air towards the needle hub, the air venting through the porous plug, the liquid reaching the porous plug and continuing down through the needle into the subcutaneous

### BRIEF DESCRIPTION OF THE DRAWINGS

FIG. 1A is a front perspective view of a vented needle hub assembly having at least one porous plug disposed in a vent in accordance with at least one embodiment of the present invention;

FIG. 1B is a front perspective view of a vented needle hub assembly having a ring of porous plug material disposed upon a plurality of vents in accordance with at least one embodiment of the present invention;

FIG. 1C is a front perspective view of a vented needle hub assembly having one or more porous plugs disposed in sidewalls (including the top) of the needle hub, and/or at the inlet of the needle hub, and/or in the flexible tubing in accordance with at least one embodiment of the present invention

FIG. 2A is a front perspective exploded view of a vented needle hub as shown in FIG. 1A in accordance with at least one embodiment of the present invention;

FIG. 2B is a front perspective exploded view of a vented needle hub as shown in FIG. 1B in accordance with at least one embodiment of the present invention;

FIG. 3 is an enlarged cut through side view of a vented needle hub in accordance with at least one embodiment of the present invention;

FIG. 4 is an enlarged cut through side view of the vented needle hub of FIG. 3, disposed into the subcutaneous tissues of a patient, and demonstrating the porous plug venting air/gas in accordance with at least one embodiment of the present invention;

FIG. 5 is an enlarged cut through side view of the vented needle hub of FIG. 3, disposed into the subcutaneous tissues of a patient, and demonstrating the porous plug having vented the air/gas and now blocking liquid which is delivered into the subcutaneous tissues of the patient in accordance with at least one embodiment of the present invention;

FIG. 6 presents a conceptual illustration of an infusion procedure utilizing at least one vented needle hub in accordance with at least one embodiment of the present invention;

FIGS. 7A-7C presents a conceptual illustration of an autoinjector as may be used in at least one embodiment for a self-priming infusion system incorporating at least one vented needle hub in accordance with at least one embodiment of the present invention;

FIG. 8A is a front perspective view of a vented needle hub assembly in a butterfly needle with flexible tubing and a connector for coupling to an autoinjector as self-priming infusion system in accordance with at least one embodiment of the present invention;

FIG. 8B is a front perspective exploded view of a vented needle hub assembly in a butterfly needle with flexible tubing and a connector for coupling to an autoinjector as self-priming infusion system in accordance with at least one embodiment of the present invention;

FIGS. 8C-8E are a plane and upper perspective views of the vented needle hub assembly from FIGS. 8A and 8B aligned to, and connected with, an autoinjector as self-priming infusion system in accordance with at least one embodiment of the present invention;

FIGS. 8F and 8G are a cut through and an enlarged view of the vented needle hub assembly coupled to an autoinjector from FIG. 8E as self-priming infusion system in accordance with at least one embodiment of the present invention;

FIG. 9A is an enlarged partial cut-through side view of the vented needle hub assembly coupled to an autoinjector as self-priming infusion system with the needle disposed into the subcutaneous tissues of a person in accordance with at least one embodiment of the present invention;

FIG. 9B is an enlarged partial cut-through side view of the vented needle hub assembly coupled to an autoinjector as self-priming infusion system with the needle disposed into the subcutaneous tissues of a person, and demonstrating the porous plug venting air/gas in accordance with at least one embodiment of the present invention;

FIG. 9C is an enlarged partial cut-through side view of the vented needle hub assembly coupled to an autoinjector as self-priming infusion system with the needle disposed into the subcutaneous tissues of a person, and demonstrating the porous plug having vented the air/gas and now blocking liquid which is delivered into the subcutaneous tissues of the patient in accordance with at least one embodiment of the present invention;

FIGS. 10A-10C are a plane and upper perspective views of the vented needle hub assembly with a luer connector aligned to, and connected with, an autoinjector as self-priming infusion system in accordance with at least one embodiment of the present invention;

FIGS. 10D and 10E are a cut through and an enlarged view of the vented needle hub assembly coupled to an autoinjector from FIG. 10C as self-priming infusion system in accordance with at least one embodiment of the present invention;

FIGS. 11A-11C are a plane and upper perspective views of the vented needle hub assembly with flexible tubing aligned to, and connected with, an autoinjector as self-priming infusion system in accordance with at least one embodiment of the present invention;

FIGS. 11D and 11E are a cut through and an enlarged view of the vented needle hub assembly coupled to an autoinjector from FIG. 11C as self-priming infusion system in accordance with at least one embodiment of the present invention;

FIGS. 12A-12C are a plane and upper perspective views of the vented needle hub assembly with a luer aligned to, and connected with, a modified autoinjector having no needle as self-priming infusion system in accordance with at least one embodiment of the present invention;

FIGS. 12D and 12E are a cut through and an enlarged view of the vented needle hub assembly coupled to an autoinjector from FIG. 12C as self-priming infusion system in accordance with at least one embodiment of the present invention;

FIG. 13 is a flow diagram of a method for providing a vented needle hub in accordance with at least one embodiment of the present invention; and

FIG. 14 is a flow diagram of a method for providing infusion therapy with at least one vented needle hub in accordance with at least one embodiment of the present invention.

### DETAILED DESCRIPTION

Before proceeding with the detailed description, it is to be appreciated that the present teaching is by way of example only, not by limitation. The concepts herein are not limited to use or application with a specific system or method for a vented needle hub for infusion without pre-priming, whether intravenous or subcutaneous, an infusion kit or other easily foreseeable alternative options or configurations where the venting of air or sterile gas is desired before a medicant or liquid is introduced to a patient. Thus, although the instrumentalities described herein are for the convenience of explanation shown and described with respect to exemplary embodiments, it will be understood and appreciated that the principles herein may be applied equally in other types of systems and methods involving a vented needle hub for infusion.

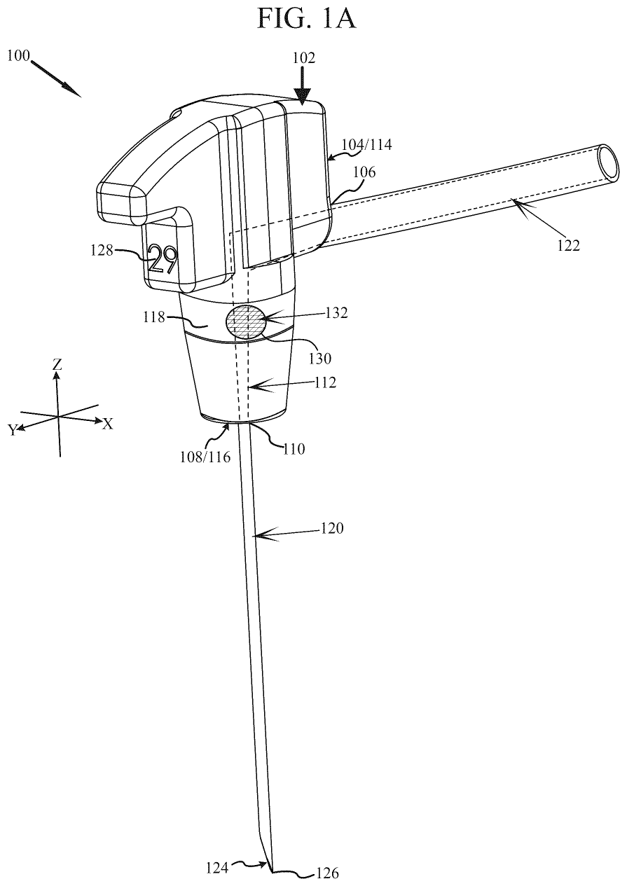

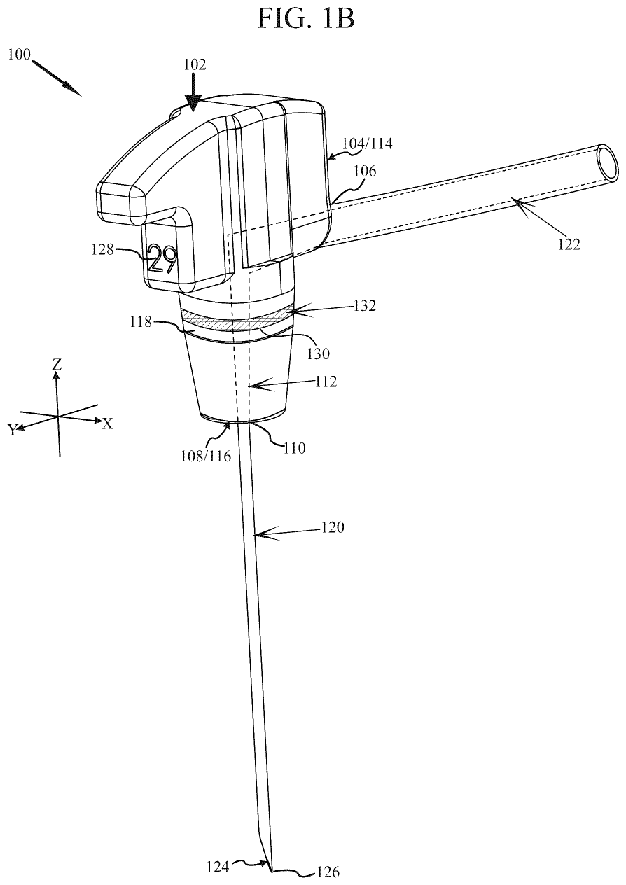

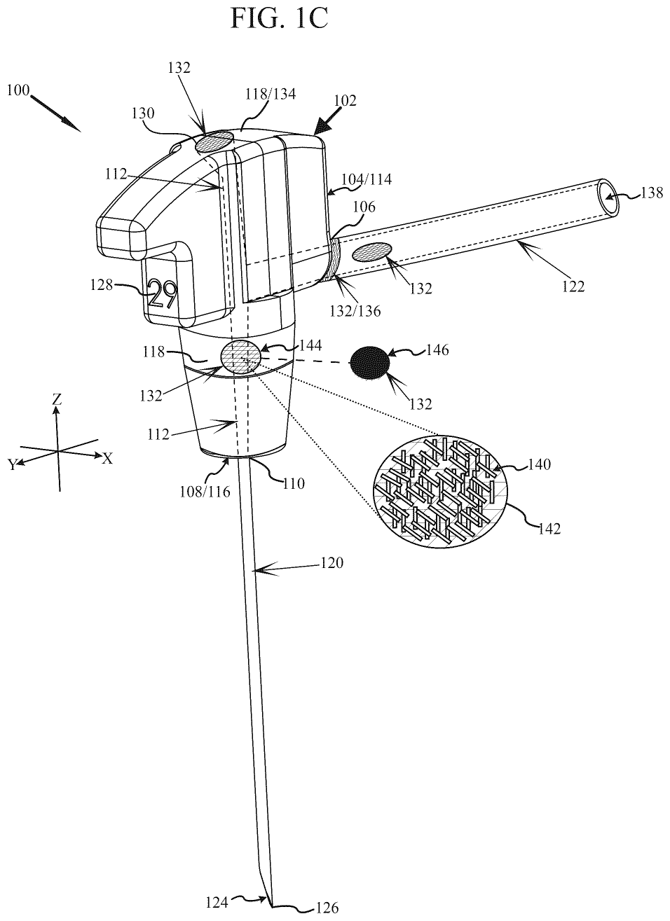

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIGS. 1A and 1B, there is shown at least one vented needle hub assembly 100, hereinafter VNH 100, in accordance with at least one embodiment of the present invention. To facilitate the description of systems and methods for embodiments of VNH 100, the orientation of VNH 100 as presented in the figures is referenced to the coordinate system with three axes orthogonal to one another as shown in FIG. 1A. The axes intersect mutually at the origin of the coordinate system, which is chosen to be the center of VNH 100, however the axes shown in all figures are offset from their actual locations for clarity and ease of illustration.

Continuing with FIG. 1A, presenting a front perspective view, for at least one embodiment, VNH 100 is generally provided by a plurality of elements, the most specific of which is perhaps the needle hub 102, having an inlet side 104, providing an inlet 106, and an outlet side 108 providing an outlet 110, and a fluid passageway 112 therebetween. The inlet side 104 may also be described as a first location 114 providing an inlet 106, and the outlet side 108 as a second location 116 providing an outlet 110.

It will also be appreciated that there is at least one sidewall 118 proximate to the fluid passageway 112 and disposed between the inlet side 104 and the outlet side 108. It will of course be understood and appreciated that the “top” of the needle hub 102 is also a sidewall 118.

An infusion needle 120 is joined to the needle hub 102, and more specifically to the outlet 110 so as to be in fluid contact with the fluid passageway 112. Similarly, flexible tubing 122 is joined to the needle hub 102, and more specifically to the inlet 106 so as to be in fluid contact with the fluid passageway 112.

As will be further discussed and described below, the infusion needle 120 is understood and appreciated to have a first length, and to be selected from a variety of different needle gauge options, such as, but not limited to, 24- to 30-gauge options. This first length will also be understood to be substantially the length of the needle protruding beyond the outlet side 108, to the opening 124 generally proximate to the distal tip 126 from which the liquid is delivered.

For at least one embodiment, the needle hub 102 may also provide indicia 128 to indicate the gage and/or length of the infusion needle 120.

As used herein, the term “flexible” as applied to the tubing 122 is understood and appreciated to mean that the tubing 122 is relatively pliable and will easily conform by bending and twisting by an operator. For at least one embodiment, the flexible tubing 122 has a luer connector structured and arranged for connection to a liquid source. Further still, for at least one embodiment the flexible tubing 122 is flow control tubing which may also be referred to as flow rate control tubing.

As shown in FIG. 1A, for at least one embodiment, the needle hub 102 receives the flexible tubing 122 and the infusion needle 120 in a configuration such that they are substantially normal to each other, such that when the needle is disposed down into subcutaneous tissue, the flexible tubing is generally parallel to the patient's skin. Moreover, for at least one embodiment it will therefore be appreciated that there is essentially a 90° transition between the flexible tubing 122 and the infusion needle 120.

In other words, for at least one embodiment the inlet 106 is normal/perpendicular to the outlet 110. It will be understood and appreciated that such a normal/perpendicular relationship between the flexible tubing 122 and the infusion needle 120 is not required for all embodiments of the present invention. Indeed, for yet another embodiment, the inlet 106 and the outlet 110 are substantially opposite or parallel to each other, such that the flexible tubing 122 and the infusion needle 120 are essentially parallel as well.

There is also at least one vent 130 disposed in the sidewall 118 of the needle hub 102, the vent in fluid contact with the fluid passageway 112. A porous plug 132 is disposed within each vent 130 adjacent to the fluid passageway 112. As shown in FIG. 1A there is one vent 130 with a porous plug 132. However, it will be appreciated that for yet another embodiment a second vent and plug may be provided on the opposite side-which is not presently visible. As noted above, the top 134 of the needle hub 102 is also a sidewall 118. For at least one embodiment the fluid passageway 112 may be extend to the top 134, such that the porous plug 132 is more easily observed by users, as is shown in FIG. 1C. Moreover, for at least one embodiment the fluid passageway 112 may be described as having a “T” or “Y” shape configuration.

As shown in FIG. 1B, for yet another embodiment, the porous plug 132 is provided as ring 132 of porous material that is disposed about a portion of the needle hub 102 so as to cover one or more vents 130 in fluid contact with the fluid passageway 112.

Returning to FIG. 1C, an additional embodiment is shown wherein the porous plug 132 is provided as an element of the inlet 106, either as one or more plug elements disposed in one or more specific vents, or as an annular ring 136 affixed to the inlet, and to which the flexible tubing 122 is directly or at least partially coupled. For such an embodiment, physical space between the end of the flexible tubing 122 and the needle hub 102 that is defined at least in part by the porous plug 132 is understood and appreciated to be the vent 132 providing gas or liquid from the flexible tubing 122 to contact the porous plug 132/136.

For yet another embodiment, the at least one vent 130 may be established in the flexible tubing 122 such that the porous plug 132 is more properly understood and appreciated to be in fluid contact with the fluid channel 138 within the flexible tubing 122 rather than the fluid passageway 112 within the needle hub 102. Of course, it is to be understood and appreciated that for at least one embodiment, there are a plurality of distinct porous plugs 132, which may be disposed in or about the sidewall 118 as well as in the top 134 as shown in FIG. 1C, and/or at or as a component of the inlet 106, and/or disposed in the flexible tubing 122.

The porous plug 132 is structured and arranged to distinguish gas from liquid. More specifically, the porous plug 132 is structured and arranged such that gas is permitted to pass from the fluid passageway through the porous plug 132 and out the at least one vent 130, but liquid is not. As used herein the terms “air” and “gas” are essentially interchangeable. When open and exposed to the natural environment, air will be permitted to enter the unsealed VNH 100. During packaging and sterilization of the VNH 100, in some embodiments, the assembled VNH 100 may be treated with an inert gas for enhanced sterilization. As such, “gas” is used herein in addition to “air” so as to convey an understanding that the present invention relates to embodiments where the VNH 100 may initially contain either “air” or some other “gas”.

For at least one embodiment, the porous plug 132 is formed at least in part of a hydrophobic material. For yet another embodiment, the porous plug 132 is formed of a plurality of micro capillary tubes 140 within a structure that would provide high surface tension to prevent liquid to pass, but still allow the passage of gas. A conceptualization of a plurality of micro capillary tubes 140 is shown in the enlarged section 142 of porous plug 132 in FIG. 1C.

Moreover, it is understood and appreciated that the porous plug 132 may be understood and appreciated as a selective vent structure, which is provided by one or more selective materials, or provided by one or more structures, or combinations of materials and structures so as to permit the passage of gas therethrough, but not liquid. More specifically, the term porous plug 132 as used herein has been adopted for convenience and ease of description, but is understood and appreciated to encompass materials and structures which in other settings might not be considered as “plug” material.

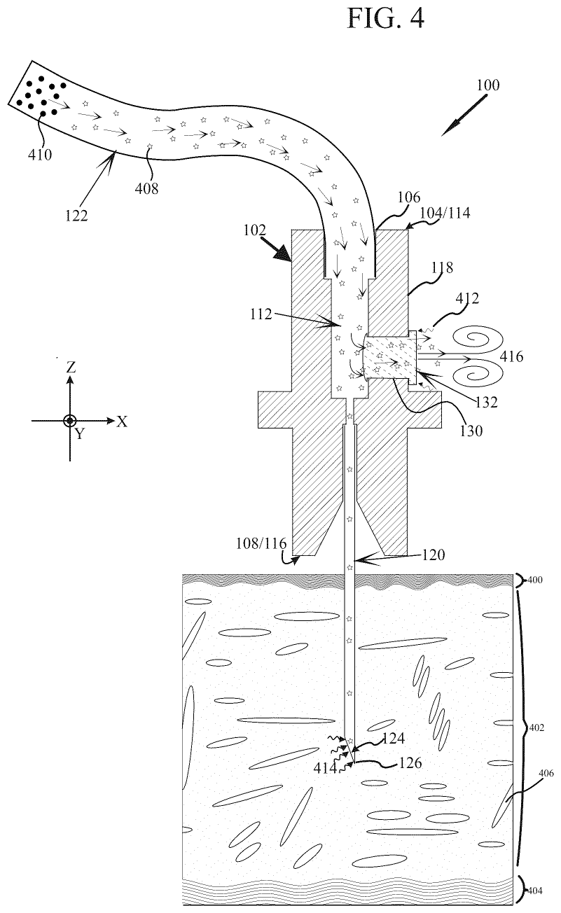

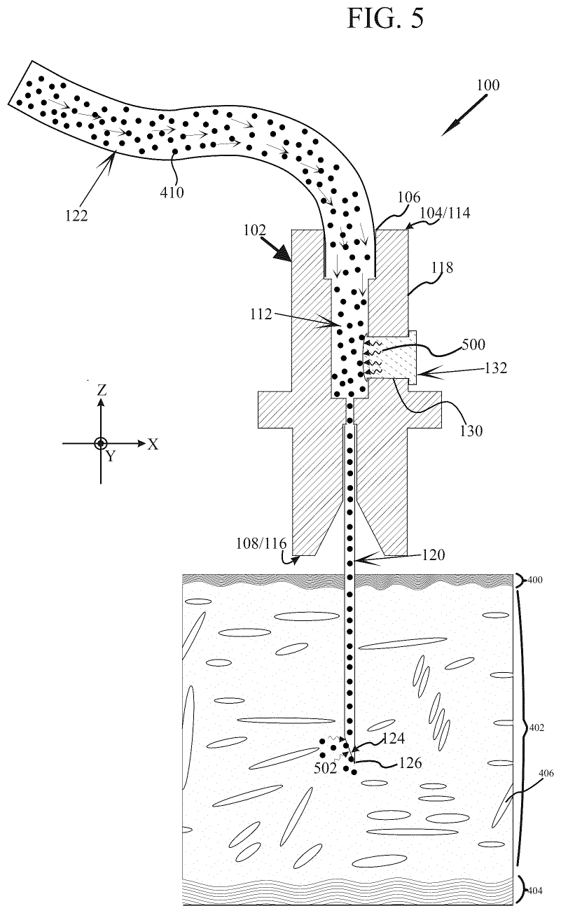

For example, in at least one embodiment, the porous plug 132 transitions to essentially a solid substance when exposed to a liquid. For example, for at least one embodiment, the porous plug 132 is provided at least in part by material that will swell when contacted by liquids, and as such pathways/channels/ducts or the like through the porous plug become even smaller and impassable to liquid, such that the porous plug 132 is a more “solid” structure then when it was dry. As will be more fully appreciated in the description below with respect to FIGS. 4 and 5, for at least one embodiment the porous plug 132 has a first air flow resistance that is lower than a second air flow resistance of subcutaneous tissue, and a first liquid flow resistance that is higher than a second liquid flow resistance of subcutaneous tissue.

In other words, for at least one embodiment, the porous plug 132 is structured and arranged such that air or other gas will more easily pass through the porous plug 132 and out of the system rather than into the subcutaneous tissues of the patient, but liquid will more easily pass into the subcutaneous tissues of the patient then through the porous plug 132 and out of the system. In truly simple terms—the porous plug 132 advantageously permits the VNH 100 to be self-priming.

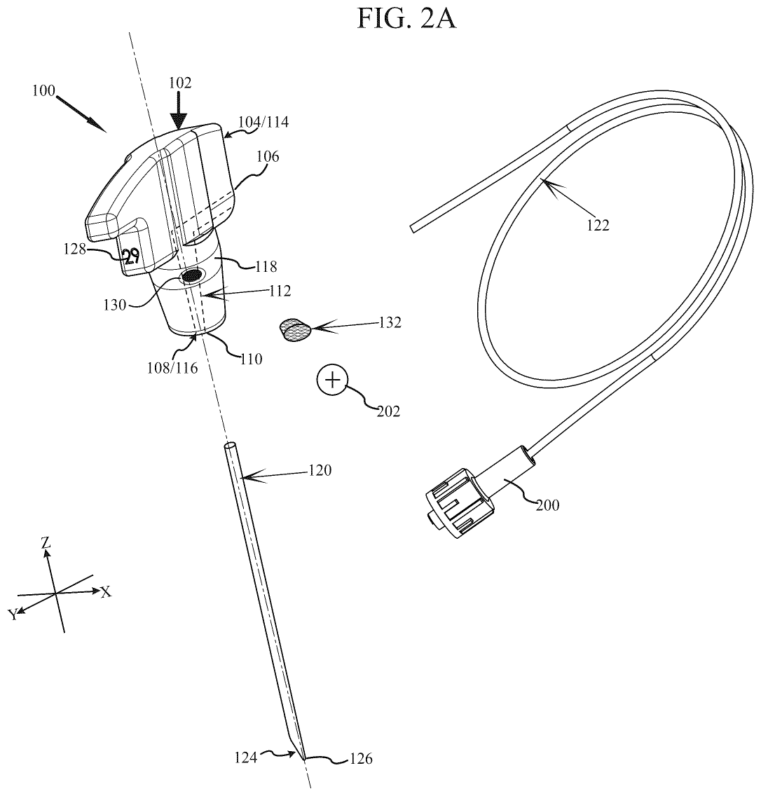

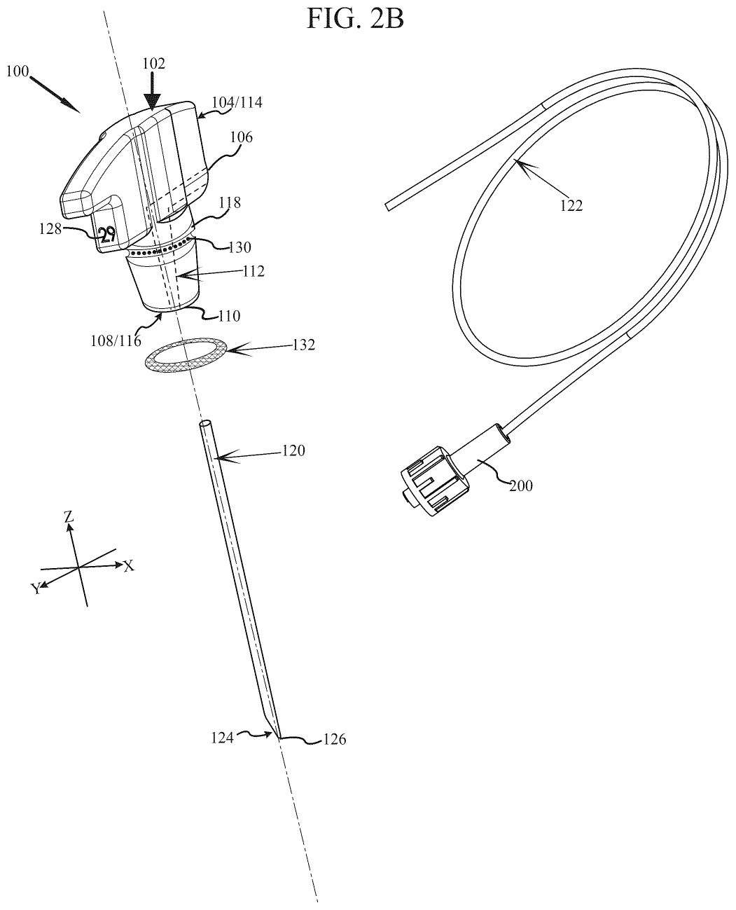

FIG. 2A provides an exploded view of the VNH 100 shown in FIG. 1A, and FIG. 2B provides an exploded view of the VNH 100 shown in FIG. 1B. In FIG. 2A, the vent 130 now exposed may be more fully appreciated, as well as the now removed and exposed porous plug 132. In FIG. 2B, the plurality of vents 130 disposed in the needle hub 102 may be appreciated with the annular ring porous plug 132 now removed.

In FIG. 2A there is also shown an optional one-way valve 202 that may be disposed over the outer surface of the porous plug 132. For at least one embodiment, the one-way valve may be a flexible membrane with one or more angled slits therein structured and arranged to open for gas passage in a first direction, but to close and seal against gas passage in a second direction. A one-way valve may also be provided for the annular ring embodiment of the porous plug 132 shown in FIGS. 1B and 2B, but for ease of illustration has not been shown.

The exploded views of FIGS. 2A & 2B also provide a conceptual representation of the flexible tubing 122, which as noted above for at least one embodiment is flow control tubing. As is also shown, for at least one embodiment the tubing 122 is coupled to a luer 200 which may be connected to a liquid reservoir or other liquid source. For at least one embodiment, the luer 200 is a flared luer as set forth in U.S. Pat. No. 10,500,389 entitled, SYSTEM AND METHOD FOR FLARED LUER

CONNECTOR FOR MEDICAL TUBING, incorporated herein by reference. For yet another embodiment, the luer 200 is a tapered luer as set forth in U.S. Provisional Application 63/616,368 entitled, SYSTEM AND METHOD FOR A TAPERED LUER CONNECTOR FOR MEDICAL TUBING, incorporated herein by reference.

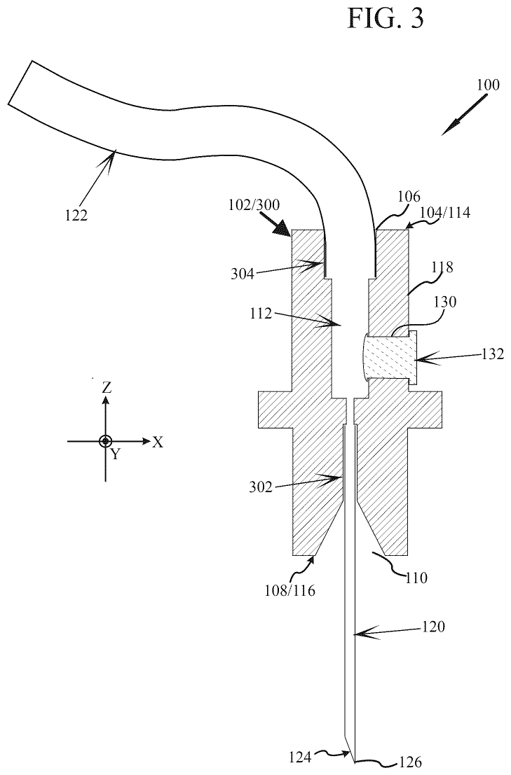

FIG. 3 provides a simplified cut through view of an exemplary embodiment of VNH 100. For ease of illustration and discussion, in this particular embedment, the inlet side 104 and the outlet side 108, and more specifically the inlet 106 and the outlet 110 have been shown to be directly opposite one another. The fluid passageway 112 between the inlet 106 and the outlet 110 may also be more fully appreciated, as well as the porous plug 132 disposed in the vent 130, such that the porous plug 132 is in fluid contact with the fluid passageway 112, but does not block or otherwise significantly impede flow as between the inlet 106 and the outlet 110.

With respect to FIG. 3, it may also be appreciated that an alternative VNH 100 is depicted, wherein the needle hub 102/300 is formed with a needle receiving section 302 such that the infusion needle 120 may be press fit, and or glued into place in a reliable and consistent fabrication process ensuring a consistent and pre-determined length of the infusion needle beyond the outlet side 108. Likewise, the needle hub 102 is formed with a flexible tubing receiving section 304 such that the end of the flexible tubing 122 may be press fit, glued, bonded or otherwise affixed into place in a reliable and consist fabrication process.

FIGS. 4 and 5 illustrate the advantageous self-priming nature of the VNH 100, with FIG. 4 first illustrating the advantageous ability of the VNH 100 to vent air/gas from the system and FIG. 5 then showing the delivery of liquid into the patient without operator adjustment to the VNH 100.

In FIG. 4, the infusion needle 120 of VNH 100 has been disposed into a patient. For ease of illustration and discussion, an enlarged portion of the patient has been rendered, showing the skin 400, and subcutaneous tissue 402, deeper muscle tissue 404 and structures such as connective tissue 406. As may be easily appreciated, the opening 124 proximate to the distal tip 126 is now disposed in the intended subcutaneous tissues 400. Moreover, the opening 124 is no longer exposed to the outside environment, e.g., air, as it is surrounded by the patient's tissues and associated liquids. It will also be appreciated that the vent 130, and more specifically the porous plug 132 is outside of the patient and therefore exposed to air or at least the gas environment in which the infusion process is being performed.

As is shown in FIG. 4, air/gas (shown as stars 408) is moving through the flexible tubing 122 as a result of liquid (shown as dots 410) being driven into the flexible tubing 122. As noted above, it may now be further appreciated that the porous plug 132 has a first air flow resistance (shown as weak arrows 412) that is lower than a second air flow resistance (shown as strong arrows 414) of the subcutaneous tissues-which results in the air/gas 402 venting 416 from the VNH 100 through the porous plug 132 rather than being forced into the subcutaneous tissues 400.

In FIG. 5, the air/gas 408 within the infusion system as shown in FIG. 4 has been substantially vented through the porous plug 132 to the point that no air/gas 408 is shown in FIG. 5 and liquid 410 has now arrived from the flexible tubing 122 into the needle hub 102. More specifically the liquid 408 has now made its way through the fluid passageway 112 and made contact with the porous plug 132. For at least one embodiment, the porous plug 132 has a first liquid flow resistance (shown as strong arrows 500) that is higher than the second liquid flow resistance (shown as weak arrows 502) or the subcutaneous tissues 400. As such, and as conceptually illustrated in FIG. 5, the liquid therefore cannot pass through the porous plug 132 and is instead directed down through the infusion needle 120 and into the subcutaneous tissues 400 of the patient.

Moreover, as conceptually illustrated by FIGS. 4 and 5, the porous plug 132 is structured and arranged to distinguish between air/gas 408 and liquid 410, air/gas 408 permitted to pass from the fluid passageway 112 through the porous plug 132 while liquid 410 is not.

For at least one embodiment this advantageous nature of the porous plug 132 is achieved at least in part by the porous plug 132 being formed at least in part from a hydrophobic material. For at least one alternative embodiment the porous plug 132 may be formed at least in part from a transitional material, structured and arranged to transition from a porous state to a solid state. More specifically, this transitional material initially in a dry state provides a plurality of channels or pathways therethrough making it porous—these channels or pathways allowing gas to pass therethrough while the transitional material is in a dry state. When exposed to a liquid containing water, the transitional material swells or otherwise transitions to a solid state and in so doing closes the channels or pathways preventing further venting of gas or liquid.

As such, it may be appreciated that the VNH 100 may be disposed into the tissues of the patient without pre-priming of the system, and therefore VNH 100 advantageously avoids issues with removing air from the infusion set, wasted liquid, potential mess, discomfort and/or pain caused by the dispersion of liquid upon the patient or about the infusion needle, and possible issues of the liquid frustrating the process of temporarily adhering the VNH 100 to the patient for the duration of the infusion treatment.

For at least one embodiment, the porous plug 132 may further include a water activated color agent. As such the porous plug 132 can and will provide a visual indication to the user and/or patient that liquid has reached the needle hub 102, as the liquid contacting the porous plug 132 causes the porous plug 132 to transition from a first color 144 to a second color 146, as shown in FIG. 1C. In other words, for at least one embodiment VNH 100, and more specifically the porous plug 132, advantageously provides visual indication that the infusion set has been primed.

It will be understood and appreciated that embodiments of VNH 100 may be advantageously incorporated in various subcutaneous infusion systems. KORU Medical Systems, Inc. of Mahwah, New Jersey, is and has been a pioneer in needle set technology and flow rate control by means of specifically engineered flow control tubing. Indeed, KORU has realized that different flow rates may be provided by working with different flow combinations of flow control tubing, such as those systems and methods set forth in U.S. Pat. No. 10,420,886 entitled MULTI-FLOW UNIVERSAL TUBING SET, incorporated herein by reference, and U.S. Pat. No. 10,709,839 entitled PRECISION VARIABLE FLOW RATE INFUSION SYSTEM AND METHOD, incorporated herein by reference.

Further, KORU has developed advantageous infusion systems permitting high flow at low pressure as set forth in U.S. application Ser. No. 17/729,914 published as US 2022/0265923 entitled HIGH FLOW AT LOW PRESSURE INFUSION SYSTEM, incorporated herein by reference. Further still, for at least one embodiment the VNH 100 is the snap in needle structure shown and described as element 162 in U.S. patent application Ser. No. 18/216,342 entitled SYSTEM AND METHOD FOR BUTTERFLY NEEDLE ASSEMBLY, incorporated herein by reference. Moreover, for at least one embodiment, the needle hub 100 is disposed in a butterfly needle assembly.

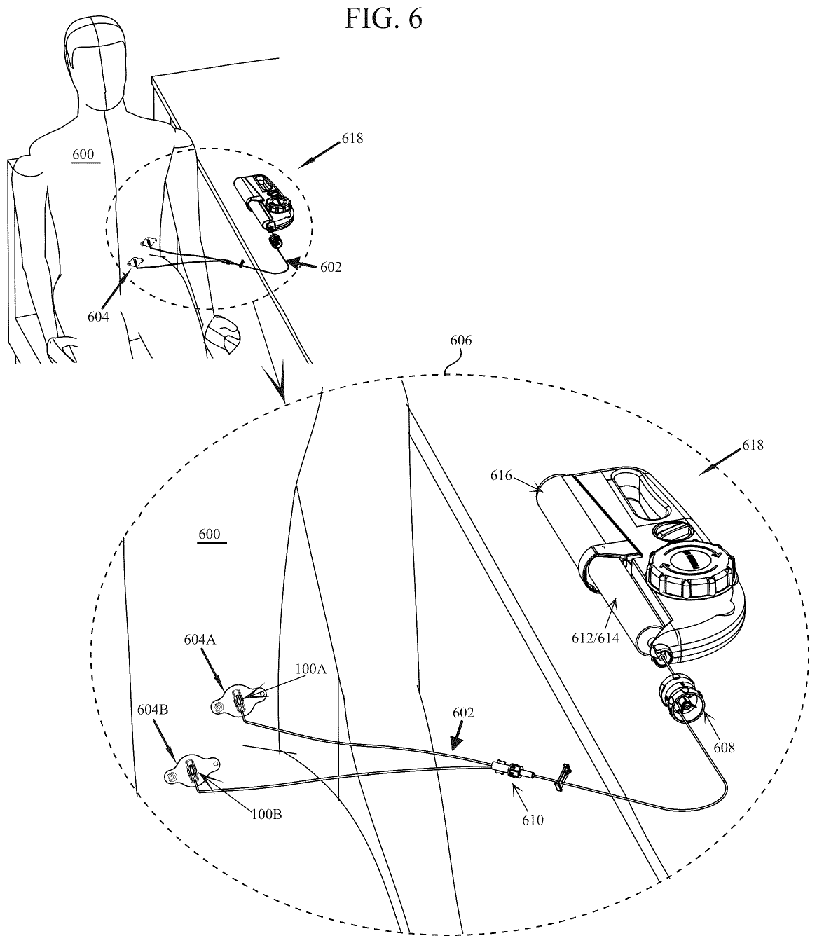

FIG. 6 presents a conceptualized infusion session for a patient 600. More specifically, a needle set 602 incorporating two butterfly needles 604A and 604B, each incorporating a VNH 100 has been disposed into a patient 600 in the lower torso area. As butterfly needles 604A and 604B have the advantageous adhesive on the generally flat bottom, no further tape is required.

As may be more fully appreciated in enlarged oval section 606, the needle set 602 is connected to a flow controller 608—this connection may be through a luer 610, or optionally the flow controller 608 may be provided as an established and inseparable element of the needle set 602. Needle set 602 is in turn connected to a liquid source or reservoir 612, such as a syringe 614.

For at least one embodiment, it is understood and appreciated that the needle set 602 is advantageously structured and arranged for use with a constant pressure pump 616, such as the Freedom60® Syringe Infusion Pump or the FreedomEdge Syringe Infusion Pump as provided by KORU Medical Systems, Inc. of Mahwah, New Jersey. Constant pressure systems, such as constant pressure pump 616, when combined with butterfly needles incorporating VNH 100, and more specifically needle sets such as needle set 602 may be highly advantageous in being self-priming to eliminate issues noted above as well as avoiding the discomfort and possible complications of introducing gas into the subcutaneous tissues.

Moreover, as set forth conceptually by FIG. 6, for at least one embodiment, needle set 602 incorporating two butterfly needles 604A and 604B, each incorporating a VNH 100 and at least pump 616 advantageously provide a self-priming system 618 for the infusion of a liquid into subcutaneous tissue.

Although the above description and illustrations have been presented with respect to subcutaneous infusion, it will be understood and appreciated that the advantageous self-priming nature of the VNH 100 to remove air from the system is advantageously adaptable to IV infusion systems and methods as well.

Moreover, with respect to the above description, at least one embodiment of VNH 100 may be summarized as a needle hub 102 having an inlet side 104 and an outlet side 108 and a fluid passageway 112 therebetween; an infusion needle 120 in fluid contact with the fluid passageway 112 and extending from the outlet side 108; a flexible tubing 122 joined to the inlet side 104 in fluid contact with the fluid passageway 112; at least one vent 130 disposed in a sidewall 118 of the needle hub 102, the vent 130 in fluid contact with the fluid passageway 112; and a porous plug 132 disposed within each at least one vent 130 adjacent to the fluid passageway, the porous plug 132 structured and arranged to distinguish gas 402 from liquid 408, gas 402 permitted to pass from the fluid passageway 112 through the porous plug 132 and out the at least one vent 130.

Further, an embodiment of VNH 100 may also be summarized as a needle hub 102 having a tubing 122 inlet at a first location and a needle 120 extending from a second location, and at least one sidewall; a fluid passageway between the tubing 122 inlet and the needle; at least one vent 130 in the needle hub 102 between the first location and the second location, the at least one vent 130 in fluid communication with the fluid passageway; and a porous plug 132 disposed within each at least one vent 130 adjacent to the fluid passageway; wherein the porous plug 132 has a first air flow resistance lower than a second air flow resistance of subcutaneous tissue, and a first liquid 408 flow resistance higher than second liquid 408 flow resistance of subcutaneous tissue.

Still yet another embodiment of VNH 100 may be summarized as a needle hub 102 having a first end and a second end and a fluid passageway 112 therebetween, the first end joined with a flexible tubing 122 line and the second end joined with a needle 120 having a first length; at least one vent 130 in the needle hub 102 between the first location and the second location, the at least one vent 130 in fluid communication with the fluid passageway; and a porous plug 132 disposed within each at least one vent 130 adjacent to the fluid passageway, the porous plug 132 structured and arranged to permit gas 402 to pass from the fluid passageway 112 through the porous plug 132 and out the at least one vent 130.

Returning to the self-priming infusion system 618 presented by FIG. 6, for yet another embodiment, VNH 100 may be coupled to a reservoir 612 that is provided by an autoinjector, thus providing an advantageous infusion system which is perhaps simpler and/or less costly than infusion system utilizing a more traditional syringe pump 616.

An autoinjector (or auto-injector or even autoinjector pen) is medical device designed to deliver a premeasured dose of medication provided in a prefilled syringe or cartridge, through a needle, without the user having to manually handle the injection steps in detail.

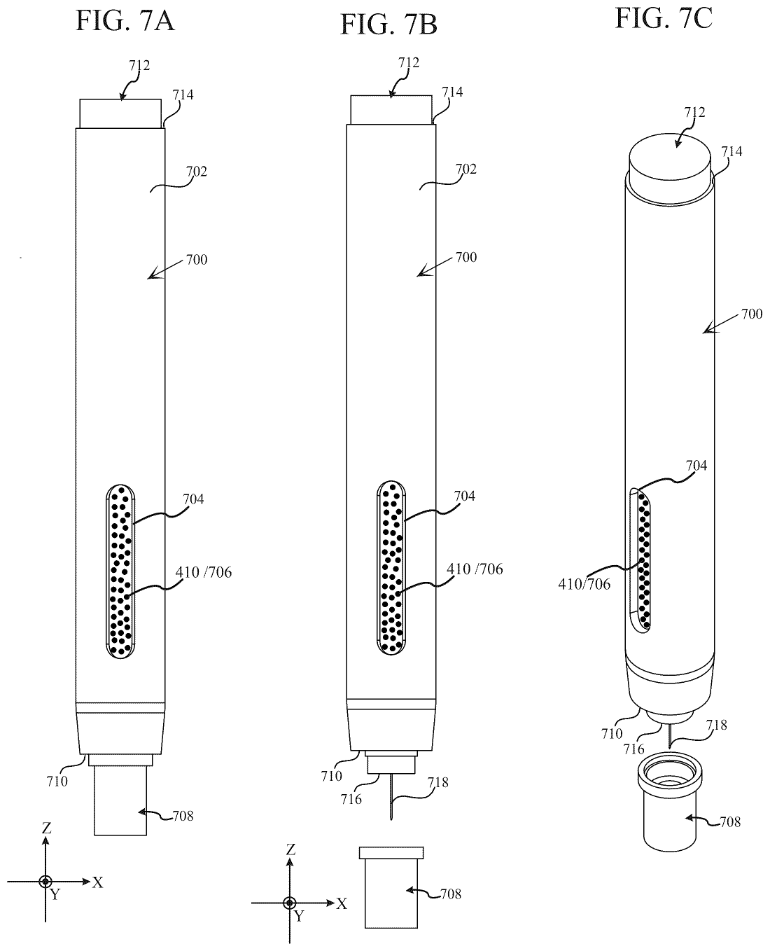

FIGS. 7A-7C show a general autoinjector 700 in an initial side view (FIG. 7A), a side view with needle exposed (FIG. 7B) and a slight upper perspective view (FIG. 7C). In general, an autoinjector 700 has an outer shell or housing 702 that encase and protects the internal syringe and needle mechanism. Commonly there is a window 704 permitting the user visual awareness of the liquid medicant, shown as dots 410, within the autoinjector 702—i.e., the reservoir 612 of liquid medicant 410 housed within the autoinjector 702.

Autoinjectors typically have a needle shield 708 disposed at a first end 710 and an activator button 712 disposed at a second end 714. With some autoinjectors, when the needle shield 708 is disposed in place at the first end 710, activation of the activator button 712 is disabled. For yet other autoinjectors, a safety trigger 716, typically in the form of a ring defining an aperture through which the needle 718 is disposed under the needle shield 708. For operation, this safety trigger 716 is at least partially disposed back into the housing 702 when the first end is disposed firmly against a patients skin, thus permitting the activation button 712 to be depressed, which in turn causes needle 718 to extend from the first end 710 (the needle 718 typically being hidden inside the housing 702 prior to activation for safety and to reduce user hesitation).

More specifically, activation of the activation button 712 triggers an internal mechanical process that in varying embodiments may utilize a spring or gas driven plunger mechanism that: a) provides force to extend the needle 718 from the housing and into the tissues of a person; and b) to expel the liquid medicant 706 through the needle 718 at a constant injection speed an volume. When the activation button is released, typically the needle 718 is retracted into the housing one again for safety and to avoid causing the user any unease.

Although some autoinjectors are refillable, many are intended to be disposable, thus simplifying out of office/clinic administration of many liquid medicants. Autoinjectors have become popular at-home options for the delivery of many kinds of drugs, such as, but certainly not limited to romosozumab and reriparatid for osteoporosis; burosumab for hypophosphatemia, epoetine alfa/darbepoetin alfa for anemia and chronic kidney diseases; sumatriptan for migraine relief; interferon beta-la and glatiramer acetate for multiple sclerosis therapies; adalimumab, enanercept, secukinumab, dupilumab and omalizumab for autoimmune and inflammatory diseases, and many others.

Although typically configured for 0.1 to 3 ml, autoinjectors are well understood technologies and may be easily preconfigured for the delivery of between about 1-20 milliliters, if not 0.1-30 milliliters, or even more. The duration of the injection process may range from a few seconds to several minutes. But even so, for some people, such as the elderly, infirm, or those with limited motor skills, the process of using an autoinjector—e.g., holding it against the skin and holding down the activator button 712, may be challenging.

Moreover, embodiments of the VNH 100 may advantageously combine with traditional autoinjectors 700 for improved ease of deliver of traditional medications for patients who have difficult with traditional operation. Further still, embodiments of the VNH 100 may advantageously combined with adapted autoinjectors 700 for infusion therapies such as Immune Globulin (Ig) therapy and the like.

Indeed, an embodiment of VNH 100 for infusion may be summarized as a needle hub 102 having an inlet side 104 and an outlet side 108 and a fluid passageway 112 therebetween; an infusion needle 120 in fluid contact with the fluid passageway 112 and extending from the outlet side 108; a flexible tubing 122 having a first end joined to the inlet side 104 in fluid contact with the fluid passageway 112, and a second end; at least one vent 130 disposed in a sidewall 118 of the needle hub 102, the vent 130 in fluid contact with the fluid passageway 112; and a porous plug 132 disposed within each at least one vent 130 adjacent to the fluid passageway 112, the porous plug 132 structured and arranged to allow gas to pass while blocking liquid, gas permitted to pass from the fluid passageway 112 through the porous plug 132 and out the at least one vent 130; and a reservoir of liquid provided as an autoinjector 700, the second end of the flexible tubing 122 interconnecting the reservoir of liquid to the inlet side 104 for fluid communication from the reservoir 700 to the needle hub 102.

This embodiment of a self-priming infusion system 800, and variations thereof, may be more fully appreciated with respect to the following figures, specifically 8A-12E.

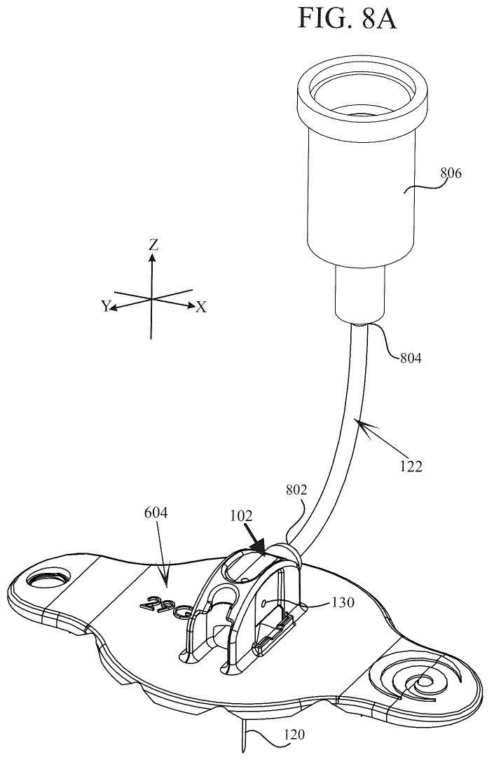

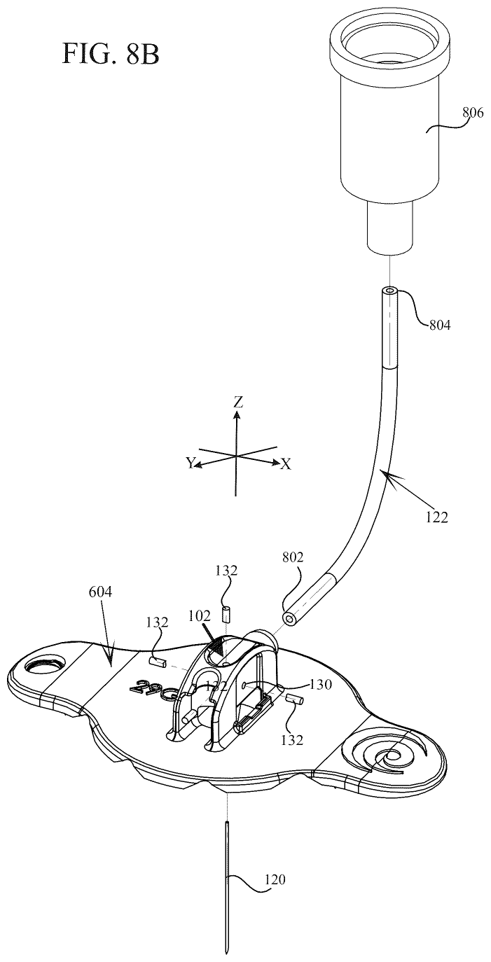

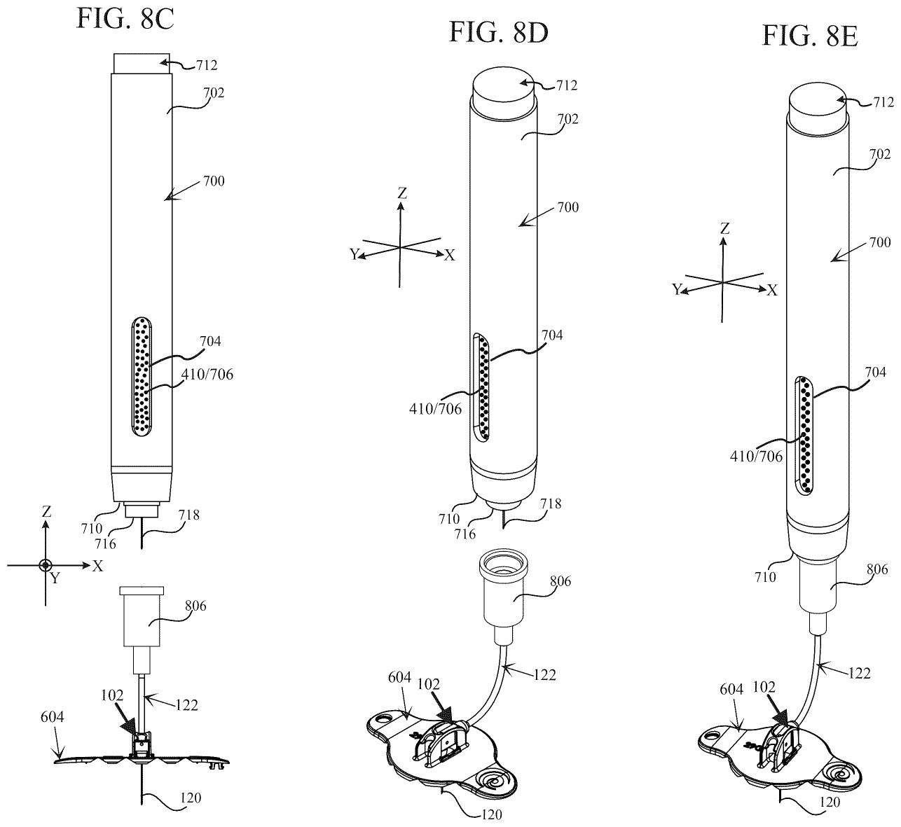

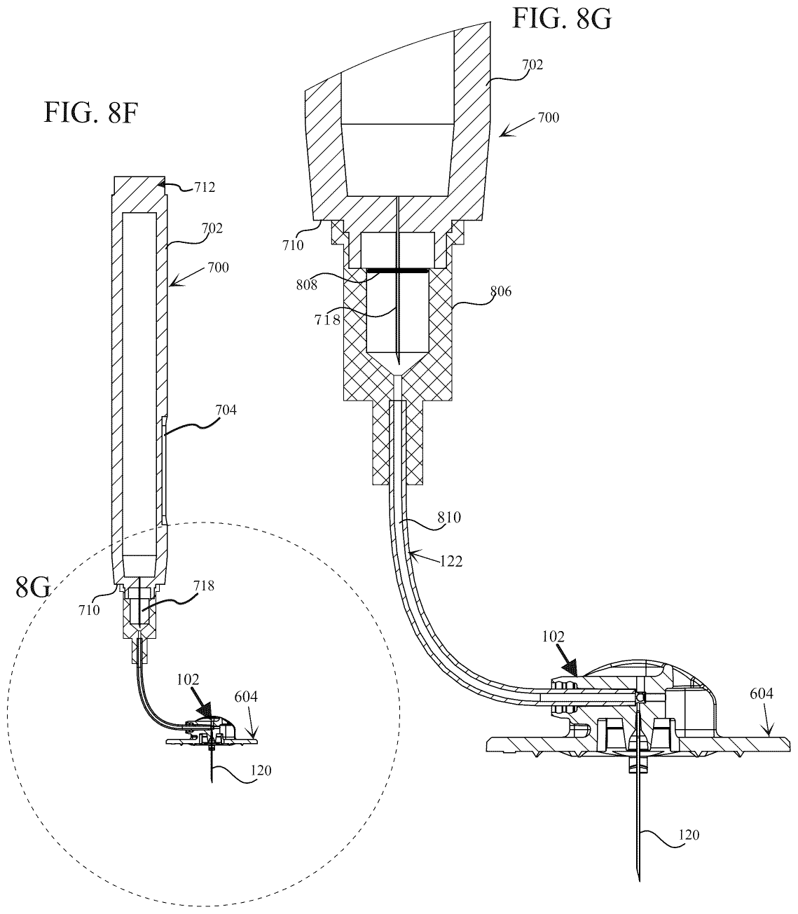

More specifically, FIG. 8A presents a perspective view of an embodiment of self-priming infusion system 800 provided by VNH 100 as a butterfly assembly 602 wherein the flexible tubing 122, has a first end 802 coupled to the inlet 106 of needle hub 102 and a second end 804 structured and arranged for coupling to the autoinjector 700. For at least one embodiment, the second end 804 of the flexible tubing is provides a connector 806, such as slip fit connector or a luer 200 for coupling to the autoinjector 700. FIG. 8B is an exploded view of FIG. 8A, further allowing appreciation of the porous plugs 132 that are disposed in the vents 130 of the needle hub 102.

The physical arrangement of autoinjector 700 and VNH 100 may be more fully appreciated in FIG. 8C, showing plane side view of the autoinjector 700 set to engage with the connector 806 of VNH 100, and FIG. 8D showing an upper perspective view of autoinjector 700 set to engage with the connector 806 of VNH 100. FIG. 8E presents an upper view of the assembled self-priming infusion system 800 provided by autoinjector 700 and VNH 100.

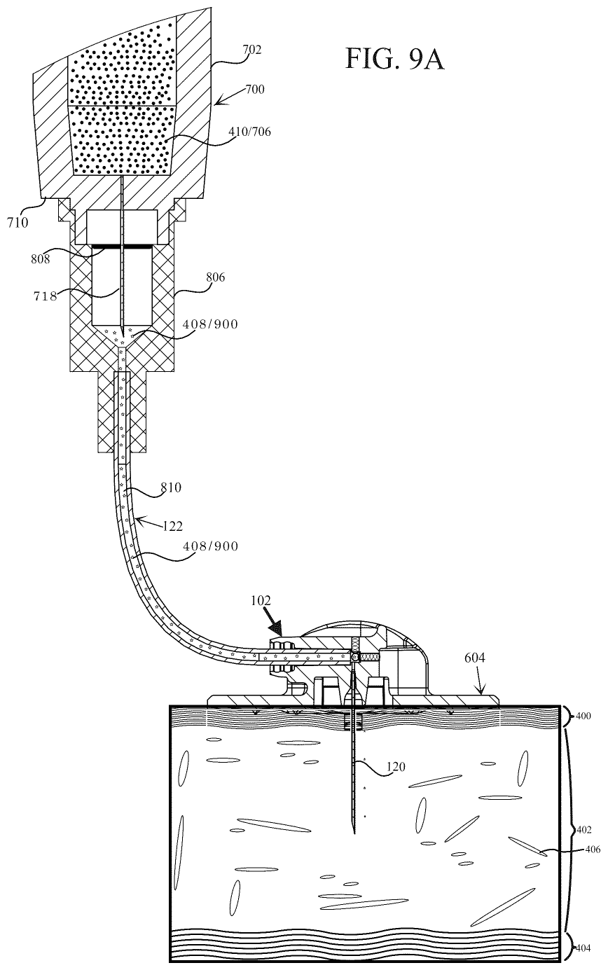

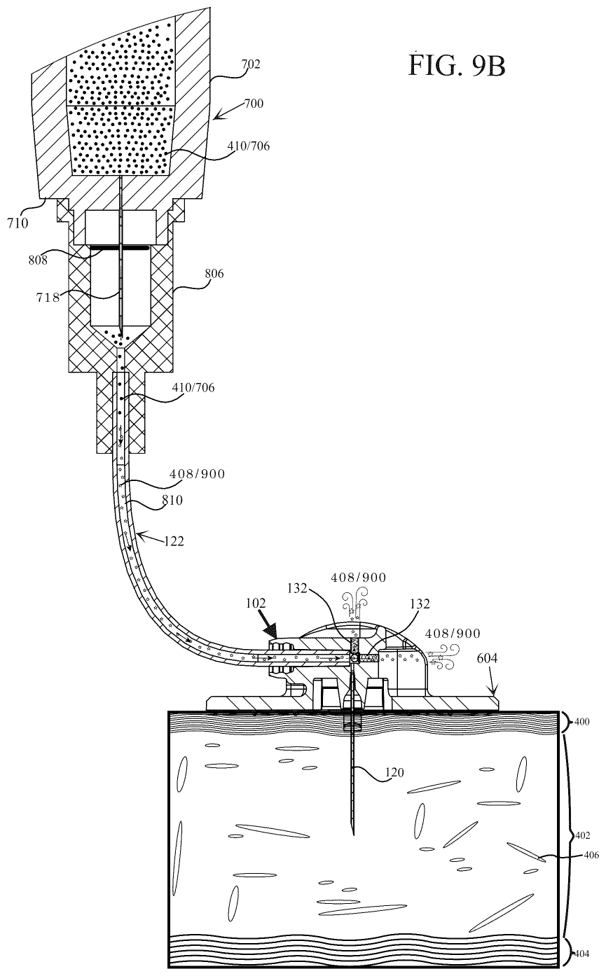

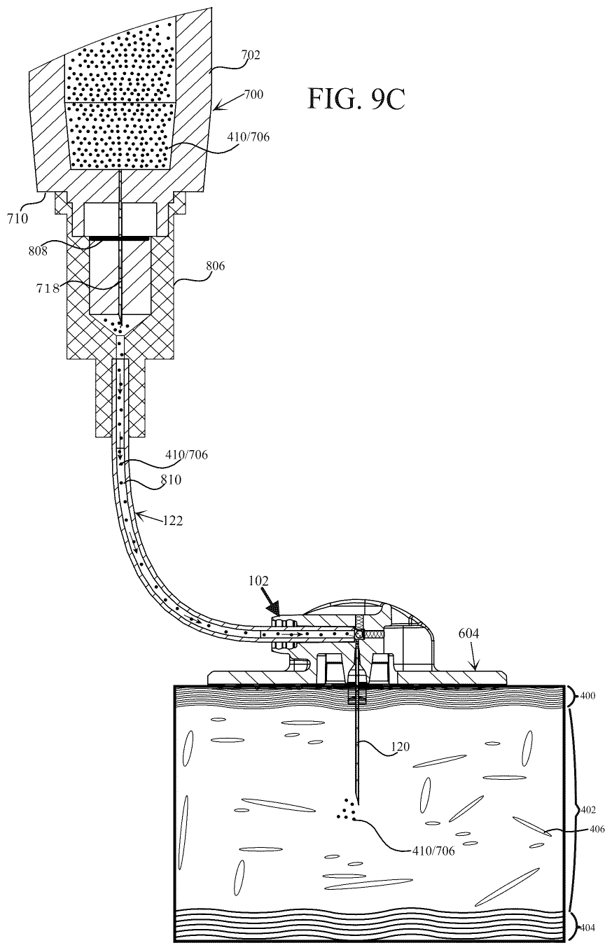

FIG. 8F presents a side cut through view of autoinjector 700 now engaged with connector 806 of VNH 100, with FIG. 8G presenting an enlarged section of FIG. 9A for further ease of discussion.

In FIGS. 8A-8D, and more specifically FIGS. 8E and 8F, it may be appreciated that the connector 800 slip fits over a portion of the first end 710 of the autoinjector 700, and more specifically the safety trigger 716. Indeed, for at least one embodiment wherein the autoinjector 700 has a safety trigger about the needle 718, the connector 806 adventitiously includes an inner abutment to depress the safety trigger 716. A frictional hold between the material of the connector 806 and the first end 710 of the autoinjector 700 holds the connector 806 in place.

For at least one embodiment, the connector 806 may be formed at least partially of silicone to provide a tacky/grippy inner surface to further assist in achieving a firm connection between the connector 806 and the first end 710, and/or safety trigger 716 of the autoinjector 700. For yet another embodiment, the connector 806 may provide an inner screw thread that is structured and arranged to engage and bind with the first end 710 and/or safety trigger 716, as the connector is twisted by a user into place on the autoinjector 700.

With respect to FIG. 8F, it will be appreciated that for at least one embodiment, the connector 806 provides a needle receiving membrane 808 that is structured and arranged to receive the needle 120 from the autoinjector 700. Prior to the needle 120 passing through the needle receiving membrane 900, the needle receiving membrane 808 provides a sterile seal to the fluid passageway 810 within the flexible tubing 122 and the fluid passageway 112 within the VNH 100.

Moreover, for at least one embodiment the flexible tubing 122 provides a connector 806 that is structured and arranged to bind with the outside of the injection end, e.g., the first end 710, of the autoinjector 700, and align the flexible tubing 122 to receive the needle 718 within the passageway 810 within the flexible tubing 122.

As may be appreciated from the figures, when needle 120 is deployed from autoinjector 700, the needle 120 passes through the membrane 808 into a sterile space proximate to the second end 802 of the tubing 122.

FIGS. 9A-9C present enlarged sections of the infusion system provided by VNH 100 with autoinjector 700 similar to FIGS. 4 and 5 discussed above, conceptually illustrating the operation of VNH 100 with autoinjector 700 as an infusion system.

In FIG. 9A, the liquid is shown as dots 410/706 and air/gas is shown as stars 408/900—the air/gas 408 initially shown to be in the needle and fluid passageway 810 of the flexible tubing 122 leading to the VNH 100. As shown in FIG. 9B, as the liquid is dispensed from the autoinjector 700, gas 900 is allowed to pass through the porous plugs 132 and out vents 130, thus evacuating the gas 900 from the tubing 122 and self-priming the VNH 100.

In FIG. 9C, as the gas has now been expelled from the system, and the porous plug 132 are structured and arranged to distinguish gas 900 from liquid 408, the liquid 408 is now directed through the needle 120 and into the subcutaneous tissues 402 of the patient.

As noted above, for at least one embodiment, the flexible tubing 122 is flow control tubing. This flexible tubing 122 also has a known length and a known internal diameter. As such the volume of the flexible tubing 122 is readily determined, and the autoinjector 700 may be pre-selected, or prefilled, to provide a sufficient reservoir of liquid 408 for the desired infusion treatment, and to prime the length of the flexible tubing 122.

With respect to embodiments of a self-priming infusion system 800, it will be understood and appreciated that different options may be preferred in different settings for the configuration of infusion system provided by the VNH 100 coupled to the autoinjector 700. As shown above in FIGS. 8A-9C, the connector 800 is structured and arranged for use with an existing/unmodified autoinjector by slip-fitting about the first end 710 of the autoinjector 700.

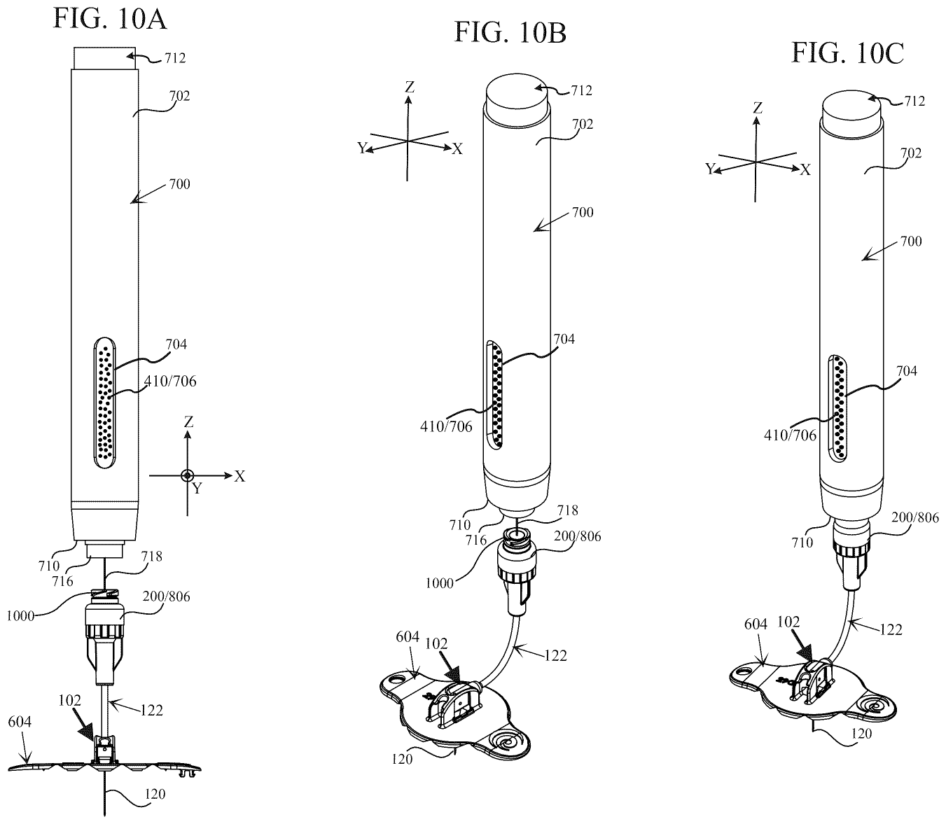

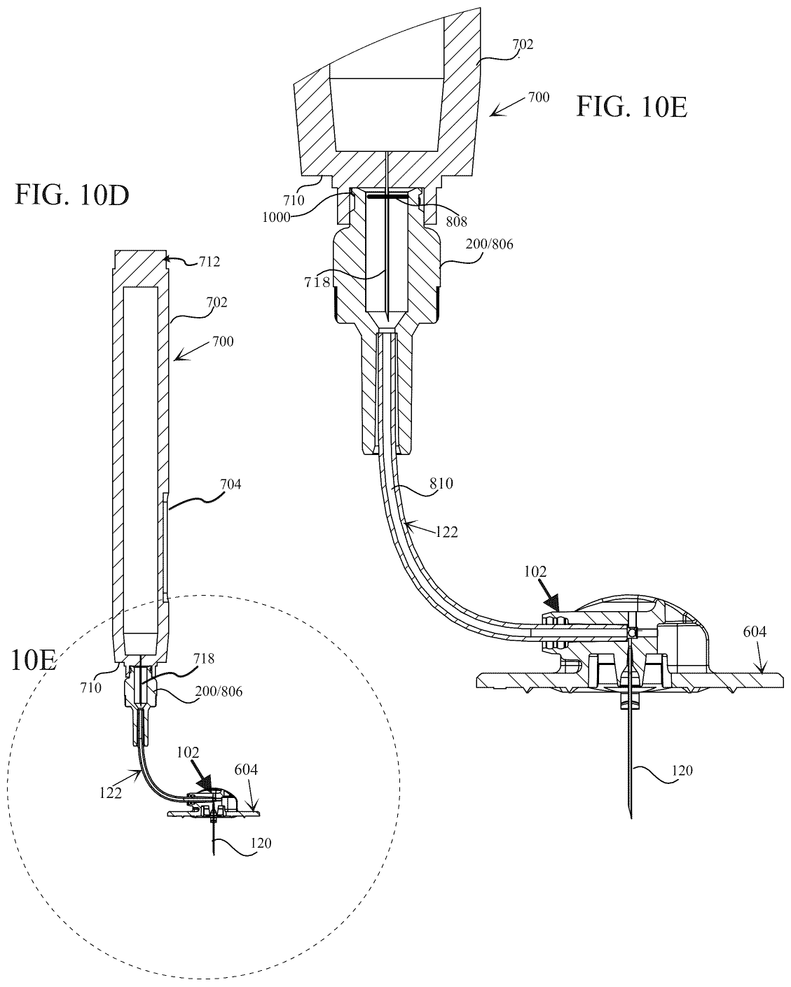

For yet another embodiment the VNH 100 connector 800 is a luer 200, providing an external thread 1000 as is shown in FIGS. 10A-10E, for yet another embodiment of self-priming infusion system 800. More specifically, FIG. 10A shows a plane side view of the autoinjector 700 set to engage with the luer 200 of VNH 100, FIG. 10B shows an upper perspective view of autoinjector 700 set to engage with luer 200 of VNH 100, and FIG. 10C shows an upper view of the assembled self-priming infusion system 800 provided by autoinjector 700 and VNH 100.

FIG. 10D presents a side cut through view of assembled self-priming infusion system 800 shown in FIG. 10C, with FIG. 10E presenting an enlarged section of FIG. 10A permitting an appreciation of the binding of the thread 1000 of the luer 200 against the inner wall of the first end 710/safety trigger 716 of the autoinjector 700. Moreover, it will be understood and appreciated that in varying embodiments of a self-priming infusion system 800, the interconnection of the second end 804 of the tubing 122 to the autoinjector may be a slip fitting, a luer connector (luer slip or luer lock), or 2-port luer as may be deemed appropriate for different types of autoinjectors 700.

Moreover, for at least one embodiment the flexible tubing 122 provides a connector 806, such as a luer 200, that is structured and arranged to bind with the inside of the injection end, e.g., the first end 710, of the autoinjector 700, and align the flexible tubing 122 to receive the needle 718 within the passageway 810 within the flexible tubing 122.

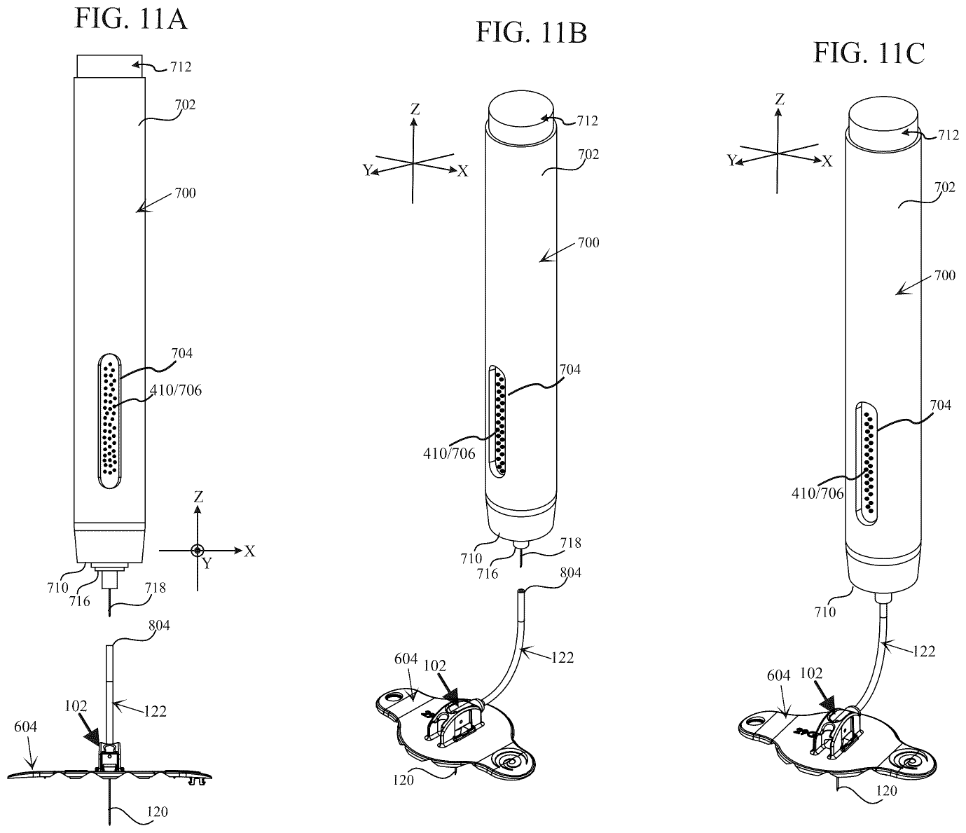

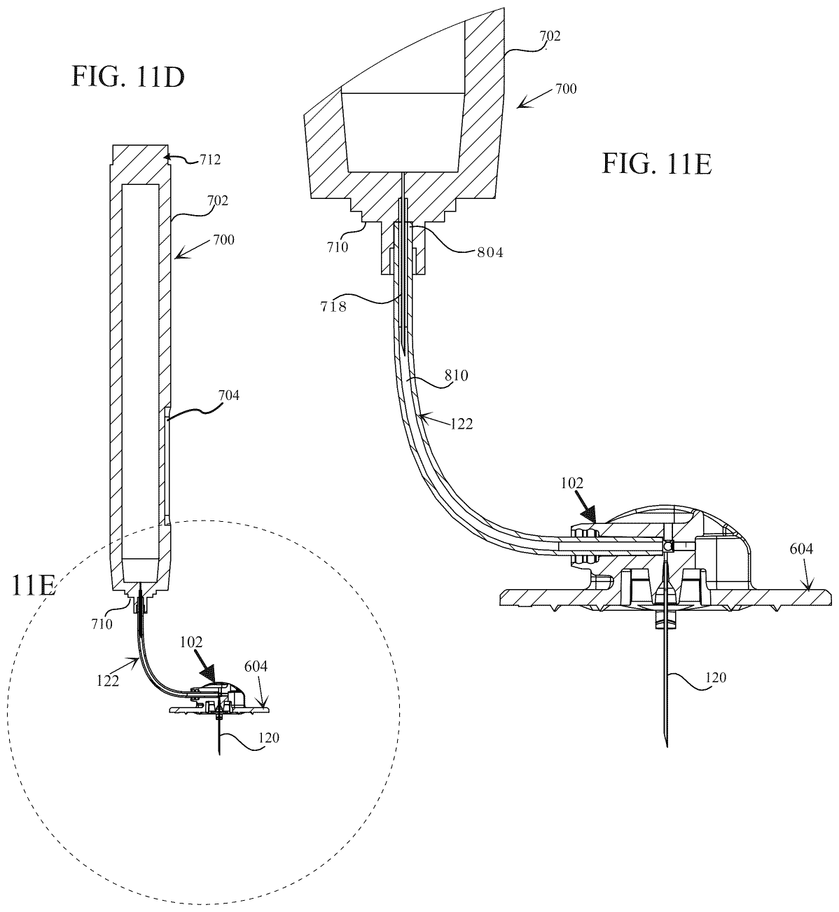

For yet another embodiment, the self-priming infusion system 800 may be provided by simply bonding the second end 804 of the flexible tubing 122 directly to the autoinjector 700 without an intervening connector 800 or luer 200. Such an embodiment is shown in FIGS. 11A-11E. More specifically, FIG. 11A shows a plane side view of the autoinjector 700 set to engage with the luer 200 of VNH 100, FIG. 11B shows an upper perspective view of autoinjector 700 set to engage with luer 200 of VNH 100, and FIG. 11C shows an upper view of the assembled self-priming infusion system 800 provided by autoinjector 700 and VNH 100.

FIG. 11D presents a side cut through view of assembled self-priming infusion system 800 shown in FIG. 11C, with FIG. 11E presenting an enlarged section of FIG. 11D permitting an appreciation of the direct bonding of the second end 804 of the tubing 122 to the autoinjector 700.

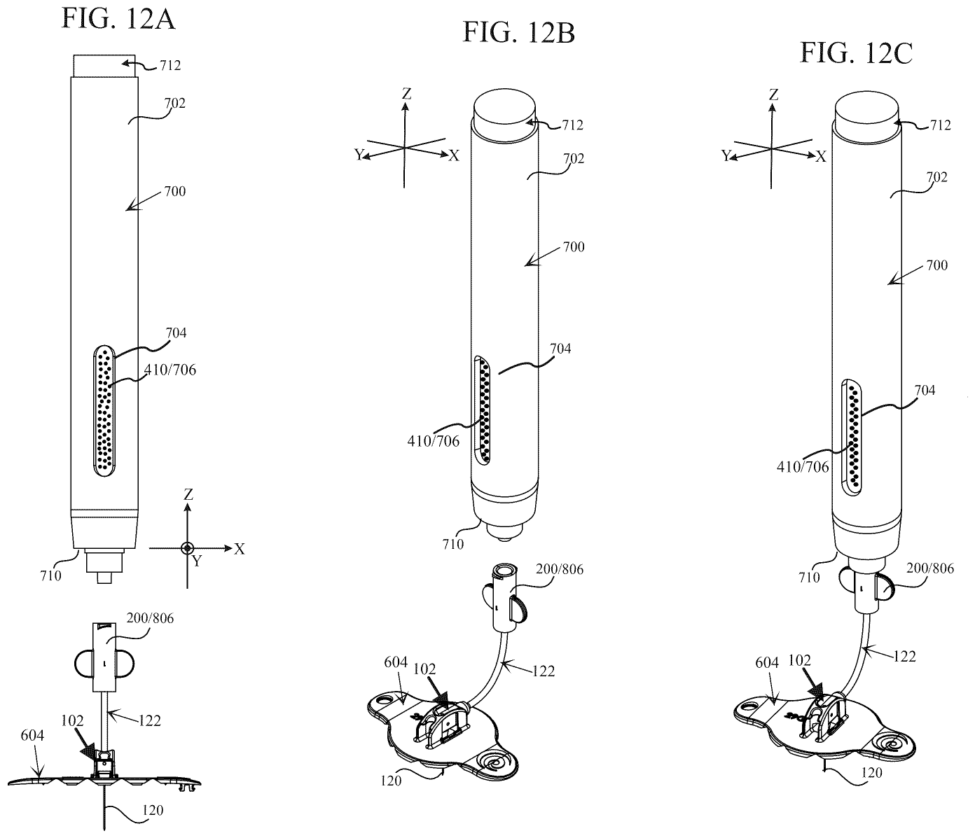

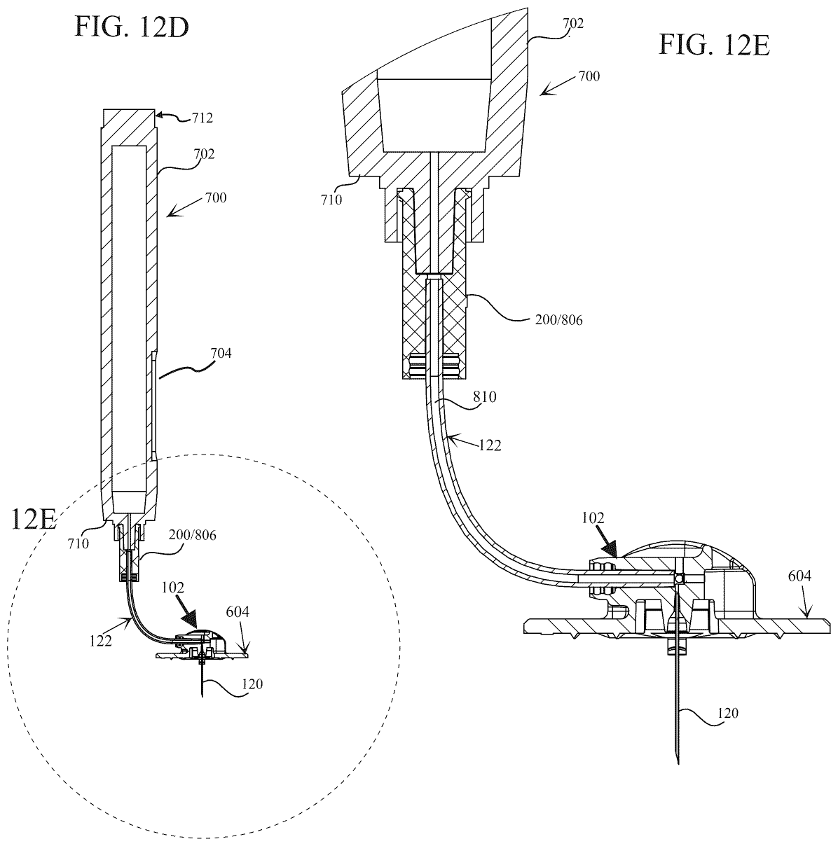

For each of the above embodiments of a self-priming infusion system 800, as illustrated in FIGS. 8C-11E, the autoinjector is essentially unmodified and the needle 718 directs liquid 408 (e.g., the liquid medicant 706) into the flexible tubing 122. For at least one alternative embodiment, the autoinjector 700 may be modified so as to have no needle. Such an embodiment is shown in FIGS. 12A-12E. More specifically, FIG. 12A shows a plane side view of the autoinjector 700 set to engage with the luer 200 of VNH 100, FIG. 12B shows an upper perspective view of autoinjector 700 set to engage with luer 200 of VNH 100, and FIG. 12C shows an upper view of the assembled self-priming infusion system 800 provided by autoinjector 700 and VNH 100.

FIG. 12D presents a side cut through view of assembled self-priming infusion system 800 shown in FIG. 12C, with FIG. 12E presenting an enlarged section of FIG. 12D permitting an appreciation of the autoinjector 700 having no needle extending into the luer 200. It will be understood and appreciated that the configuration conceptually illustrated in FIGS. 11A-11E wherein the tubing 122 is directed bonded to the autoinjector 700 may likewise be modified to utilize an autoinjector 700 that has been modified to remove the needle.

Indeed, it will be understood and appreciated that for the embodiments shown and described in FIGS. 8A-10E and 12A-12E the VNH 100 with flexible tubing 122 and connector 806 may be provided as a unitary structure that is separate from the autoinjector 700, wherein a user then assembles the self-priming system infusion system 800 by removing each element from sterile packaging and then connecting them. For yet other embodiments, as with the embodiment shown in FIGS. 11A-11E wherein the second end of the flexible tubing is bonded to the autoinjector 700, the VNH 100 with flexible tubing 122 and connector 806 of the embodiments shown in shown and described in FIGS. 8A-10E and 12A-12E may be bonded, joined, or otherwise affixed to the autoinjector 700 to provide the self-priming system infusion system 800 as an entire unified and contiguous system.

With respect to the above embodiments incorporating a connector 800 or a luer 200, for yet another embodiment it will be understood and appreciated that such a connector 800 or luer 200 may be structured and arranged to provide a flange or flaring (not shown) that faces away from the autoinjector 700. For such embodiments, the flexible tubing 122 may be initially coiled and disposed substantially within the flange with or without a breakaway/tear-away covering so as to provide a neat and compact form of the VNH 100 assembly.

Moreover, an embodiment of VNH 100 incorporated to provide a self-priming system for the infusion of liquid into subcutaneous tissues may be summarized as a needle hub 102 having a tubing inlet 106 at a first location 114 and a needle 120 extending from a second location 116, and at least one sidewall 118; a fluid passageway 112 between the tubing inlet 106 and the needle 120; at least one vent 130 in the needle hub 102 between the first location 114 and the second location 116, the at least one vent 130 in fluid communication with the fluid passageway 112; and a porous plug 132 disposed within each at least one vent 130 adjacent to the fluid passageway 112; and a reservoir of liquid provided as an autoinjector 700; a flexible tubing 122 having a first end coupled to the tubing inlet 106 and a second end for interconnecting the reservoir of liquid 700 to the tubing inlet 106 for fluid communication from the reservoir 700 to the needle hub 102; wherein the porous plug 132 has a first air flow resistance 412 lower than a second air flow resistance 414 of subcutaneous tissue 402, and a first liquid flow resistance higher than a second liquid flow resistance of subcutaneous tissue 402.

Yet another embodiment of VNH 100 incorporated to provide a self-priming system for the infusion of liquid into subcutaneous tissues may be summarized as a needle hub 102 having a first end 104 and a second end 108 and a fluid passageway 112 therebetween, the first end 104 joined with a first end of a flexible tubing line 122 and the second end 108 joined with a needle 120; at least one vent 130 in the needle hub 102 between the first end 104 and the second end 108, the at least one vent 130 in fluid communication with the fluid passageway 112; a porous plug 132 disposed within each at least one vent 130 adjacent to the fluid passageway 112, the porous plug 132 structured and arranged to permit gas 900 to pass from the fluid passageway 112 through the porous plug 132 and out the at least one vent 130; a reservoir of liquid provided as an autoinjector 700, the flexible tubing 122 having a second end for interconnecting the reservoir of liquid 700 to the needle hub 102 for fluid communication from the reservoir 700 to the needle hub 102.

Having described embodiments of the VNH 100, other embodiments relating to at least one method of providing a VNH 100, as well as at least one method of providing infusion therapy into subcutaneous tissues with a VNH 100 will now be discussed. It will be appreciated that the described methods need not be performed in the order in which they are herein described, but that these descriptions are merely exemplary of methods to provide and use a VNH 100.

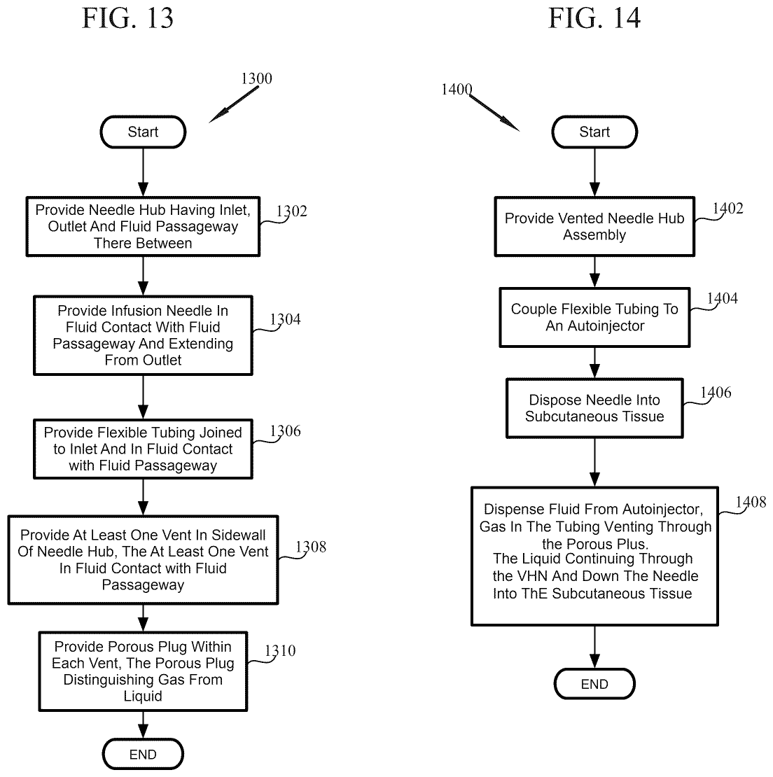

Turning to FIG. 13, presented is a flow diagram for at least one method 1300 of providing a VNH 100. In general, method 1300 commences with providing a needle hub 102 having an inlet 106 and an outlet 110 and a fluid passageway 112 therebetween, block 1302. As discussed above, for at least one embodiment, the inlet 106 is provided by an inlet side 104 and the outlet is provided by an outlet side 108.

An infusion needle 120 is also provided in fluid contact with the fluid passageway 112 and extending from the outlet side, block 1304. A flexible tubing 122 is also provided and joined to the inlet 106 of the inlet side 106 in fluid contact with the fluid passageway 112, block 1306. At least one vent 130 is also provided in the sidewall of the needle hub, the vent 130 in fluid communication with the fluid passageway 112, block 1308. Of course, it will be understood and appreciated that for at least one embodiment, the at least one vent may be established during the process of forming the needle hub 102 such as by 3D printing, stamping, casting or such other fabrication process.

Method 1300 concludes with providing a porous plug 132 in each of the provided vents 130, the porous plug 132 structured and arranged to distinguish gas from liquid, gas permitted to pass from the passageway 112 through the porous plug 132 and out the at least one vent 130, block 1310.

Moreover, method 1300 may be summarized as providing a needle hub 102 having an inlet side 104 and an outlet side 108 and a fluid passageway 112 therebetween; providing an infusion needle in fluid contact with the fluid passageway 112 and extending from the outlet side 108; providing a flexible tubing 122 joined to the inlet side 104 in fluid contact with the fluid passageway 112; providing at least one vent 130 disposed in a sidewall 118 of the needle hub 102, the vent 130 in fluid contact with the fluid passageway 112; providing a porous plug 132 disposed within each at least one vent 130 adjacent to the fluid passageway, the porous plug 132 structured and arranged to distinguish gas 402 from liquid 404, gas 402 permitted to pass from the fluid passageway 112 through the porous plug 132 and out the at least one vent 130.

Turning to FIG. 14, presented is a flow diagram for at least one method 1400 of providing infusion therapy with a VNH 100, and more specifically a self-priming system 800 for infusion of a liquid into subcutaneous tissues. In general, method 1400 commenced with providing a VNH 100 in accordance with at least one of the embodiments discussed and described above, block 1402.

The flexible tubing is coupled to a liquid reservoir, such as an autoinjector 700 shown in FIG. 7, block 1404. It will be understood and appreciated that in the initial starting state, there is some quantity of air or gas in the VNH 100. Method 1400 continues with disposing the needle into the subcutaneous tissue of a patient, block 1406. Now, with the needle properly disposed into the subcutaneous tissues of the patient, the infusion method 1400 continues with the dispensing of the liquid from the autoinjector 700 into the flexible tubing 122 which in turn drives the air/gas within the tubing towards the needle hub 102, block 1408.

As described above, as the porous plug 132 is structured and arranged to distinguish between gas and liquid, this air/gas advantageously vents through the porous plug 132. As the liquid arrives within the needle hub 102, the porous plug 132 does not permit the liquid to pass through and out the vent 130, and the liquid is therefore further directed through the infusion needle 120 and into the subcutaneous tissue, block 1408.

Moreover, method 1400 may be summarized as providing an infusion system comprising: a needle hub 102 having an inlet side 104 and an outlet side 108 and a fluid passageway 112 therebetween; an infusion needle 120 in fluid contact with the fluid passageway 112 and extending from the outlet side 108; a flexible tubing 122 having a first end joined to the inlet side 104 in fluid contact with the fluid passageway 112, and a second end; at least one vent 130 disposed in a sidewall 118 of the needle hub 102, the vent 130 in fluid contact with the fluid passageway 112; and a porous plug 132 disposed within each at least one vent 130 adjacent to the fluid passageway 112, the porous plug 132 structured and arranged to distinguish gas 900 from liquid, gas 900 permitted to pass from the fluid passageway 112 through the porous plug 132 and out the at least one vent 130; and a reservoir of liquid provided as an autoinjector 700, the second end of the flexible tubing 122 interconnecting the reservoir of liquid 700 to the inlet side 104 for fluid communication from the reservoir 700 to the needle hub 102; disposing the needle 120 into the subcutaneous tissue 402 of a patient 600; and activating the autoinjector 700 to dispense liquid 706 from the reservoir 700 into the flexible tubing 122, the liquid 706 propagating the air 408/900 towards the needle hub 102, the air venting through the porous plug 132, the liquid 706 reaching the porous plug 132 and continuing down through the needle 120 into the subcutaneous tissues 402.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A self-priming system for the infusion of a liquid into subcutaneous tissue, comprising:
a needle hub having an inlet side and an outlet side and a fluid passageway therebetween;
an infusion needle in fluid contact with the fluid passageway and extending from the outlet side;
a flexible tubing having a first end joined to the inlet side in fluid contact with the fluid passageway, and a second end;
at least one vent disposed in a sidewall of the needle hub, the vent in fluid contact with the fluid passageway; and
a porous plug disposed within each at least one vent adjacent to the fluid passageway, the porous plug structured and arranged to allow gas to pass while blocking liquid, gas permitted to pass from the fluid passageway through the porous plug and out the at least one vent; and
a reservoir of liquid provided as an autoinjector, the second end of the flexible tubing interconnecting the reservoir of liquid to the inlet side for fluid communication from the reservoir to the needle hub.

2. The self-priming system of claim 1, wherein the second end of the tubing provides a luer connector for coupling to the autoinjector.

3. The self-priming system of claim 1, wherein the second end of the tubing provides a needle receiving membrane, structured and arranged to receive a needle from the autoinjector, the membrane a sterile seal to a fluid passageway within the flexible tubing.

4. The self-priming system of claim 1, wherein the autoinjector has an injection end with an outside, the second end of the tubing provides a luer connector structured and arranged to bind with the outside of injection end, and align the flexible tubing to receive a needle within a passageway within the tubing.

5. The self-priming system of claim 1, wherein the autoinjector has an injection end with an inside, the second end of the tubing provides a luer connector structured and arranged to bind with the inside of injection end, and align the flexible tubing to receive a needle within a passageway within the tubing.

6. The self-priming system of claim 1, wherein the second end of the tubing is joined to the autoinjector during fabrication.

7. The self-priming system of claim 1, wherein the flexible tubing has a known length, the autoinjector providing a sufficient reservoir of liquid for the desired infusion treatment, and to prime the length of flexible tubing.

8. The self-priming system of claim 1, wherein the vented needle hub assembly is disposed in a butterfly needle assembly.

9. A self-priming system for the infusion of a liquid into subcutaneous tissue, comprising:
a needle hub having a tubing inlet at a first location and a needle extending from a second location, and at least one sidewall;
a fluid passageway between the tubing inlet and the needle;
at least one vent in the needle hub between the first location and the second location, the at least one vent in fluid communication with the fluid passageway; and
a porous plug disposed within each at least one vent adjacent to the fluid passageway; and
a reservoir of liquid provided as an autoinjector;
a flexible tubing having a first end coupled to the tubing inlet and a second end for interconnecting the reservoir of liquid to the tubing inlet for fluid communication from the reservoir to the needle hub;
wherein the porous plug has a first air flow resistance lower than a second air flow resistance of subcutaneous tissue, and a first liquid flow resistance higher than a second liquid flow resistance of subcutaneous tissue.

10. The self-priming system of claim 9, wherein the second end of the tubing provides a luer connector for coupling to the autoinjector.

11. The self-priming system of claim 9, wherein the second end of the tubing provides a needle receiving membrane, structured and arranged to receive a needle from the autoinjector, the membrane a sterile seal to a fluid passageway within the flexible tubing.

12. The self-priming system of claim 9, wherein the autoinjector has an injection end, the second end of the tubing provides a luer connector structured and arranged to bind with the outside of injection end, and align the flexible tubing to receive a needle within a passageway within the tubing.

13. The self-priming system of claim 9, wherein the second end of the tubing is joined to the autoinjector during fabrication.

14. The self-priming system of claim 9, wherein the flexible tubing has a known length, the autoinjector providing a sufficient reservoir of liquid for the desired infusion treatment, and to prime the length of flexible tubing.

15. The self-priming system of claim 9, wherein the vented needle hub assembly is disposed in a butterfly needle assembly.

16. A self-priming system for the infusion of a liquid into subcutaneous tissue, comprising:
a needle hub having a first end and a second end and a fluid passageway therebetween, the first end joined with a first end of a flexible tubing line and the second end joined with a needle;
at least one vent in the needle hub between the first end and the second end, the at least one vent in fluid communication with the fluid passageway;
a porous plug disposed within each at least one vent adjacent to the fluid passageway, the porous plug structured and arranged to permit gas to pass from the fluid passageway through the porous plug and out the at least one vent;
a reservoir of liquid provided as an autoinjector, the flexible tubing having a second end for interconnecting the reservoir of liquid to the needle hub for fluid communication from the reservoir to the needle hub.

17. The self-priming system of claim 16, wherein the second end of the tubing provides a luer connector for coupling to the autoinjector.

18. The self-priming system of claim 16, wherein the second end of the tubing provides a needle receiving membrane, structured and arranged to receive a needle from the autoinjector, the membrane a sterile seal to a fluid passageway within the flexible tubing.

19. The self-priming system of claim 16, wherein the autoinjector has an injection end with an inside, the second end of the tubing provides a luer connector structured and arranged to bind with the inside of injection end, and align the flexible tubing to receive a needle within a passageway within the tubing.

20. The self-priming system of claim 16, wherein the autoinjector has an injection end with an outside, the second end of the tubing provides a luer connector structured and arranged to bind with the outside of injection end, and align the flexible tubing to receive a needle within a passageway within the tubing.

21. The self-priming system of claim 16, wherein the second end of the tubing is joined to the autoinjector during fabrication.

22. The self-priming system of claim 16, wherein the flexible tubing has a known length, the autoinjector providing a sufficient amount of liquid for the desired infusion treatment, and to prime the length of flexible tubing.

23. The self-priming system of claim 16, wherein the vented needle hub assembly is disposed in a butterfly needle assembly.

24. A method of using a self-priming system for the infusion of a liquid into subcutaneous tissue for infusion, comprising:
providing an infusion system comprising:
a needle hub having an inlet side and an outlet side and a fluid passageway therebetween;
an infusion needle in fluid contact with the fluid passageway and extending from the outlet side;
a flexible tubing having a first end joined to the inlet side in fluid contact with the fluid passageway, and a second end;
at least one vent disposed in a sidewall of the needle hub, the vent in fluid contact with the fluid passageway; and
a porous plug disposed within each at least one vent adjacent to the fluid passageway, the porous plug structured and arranged to distinguish gas from liquid, gas permitted to pass from the fluid passageway through the porous plug and out the at least one vent; and
a reservoir of liquid provided as an autoinjector, the second end of the flexible tubing interconnecting the reservoir of liquid to the inlet side for fluid communication from the reservoir to the needle hub;
disposing the needle into the subcutaneous tissue of a patient; and
activating the autoinjector to dispense liquid from the reservoir into the flexible tubing, wherein the liquid propagates the air towards the needle hub, the air venting through the porous plug, the liquid reaching the porous plug and continuing down through the needle into the subcutaneous tissues.

25. The method of claim 24, wherein the second end of the tubing provides a luer connector for coupling to the autoinjector.

26. The method of claim 24, wherein the second end of the tubing provides a needle receiving membrane, structured and arranged to receive a needle from the autoinjector, the membrane a sterile seal to a fluid passageway within the flexible tubing.

27. The method of claim 24, wherein the autoinjector has an injection end, the second end of the tubing provides a luer connector structured and arranged to bind with the injection end, and align the flexible tubing to receive a needle within a passageway within the tubing.

28. The method of claim 24, wherein the second end of the tubing is joined to the autoinjector during fabrication.

29. The method of claim 24, wherein the flexible tubing has a known length, the autoinjector providing a sufficient amount of liquid for the desired infusion treatment and to prime the length of flexible tubing.

30. The method of claim 24, wherein the vented needle hub assembly is disposed in a butterfly needle assembly.
